(19) United States
(12) Patent Application Publication
(10) Pub. No.: US 2024/0230642 A1
(43) Pub. Date: Jul. 11, 2024

(54) METHODS AND KITS FOR DIAGNOSING SYSTEMIC AUTOIMMUNE RHEUMATIC DISEASES

(71) Applicant: Université Laval, Québec (CA)

(72) Inventors: ÉRIC BOILARD, Québec (CA); Paul R. Fortin, Quebec (CA); Yann Becker, Quebec (CA)

(21) Appl. No.: 18/563,723

(22) PCT Filed: May 26, 2022
(86) PCT No.: PCT/CA2022/050849

§ 371 Date: Nov. 22, 2023

Related U.S. Application Data
(60) Provisional application No. 63/202,080, filed on May 26, 2021.

(51) Int. Cl. G01N 33/564; A61K 45/06; C07K 17/00; G01N 33/68

(52) U.S. Cl. CPC G01N 33/564; A61K 45/06; C07K 17/00; G01N 33/6854; G01N 2333/4716; G01N 2800/104; G01N 2800/56

(57) ABSTRACT

Described herein are methods and kits for diagnosing or determining a progression of systemic autoimmune rheumatic diseases, comprising determining levels of autoantibodies specific to one or more mitochondrial proteins.

The present description relates to a method and kits for diagnosing or determining the progression of systemic autoimmune rheumatic diseases, comprising determining levels of autoantibodies specific to one or more mitochondrial proteins.

The present description refers to a number of documents, the contents of which are herein incorporated by reference in their entirety.

### BACKGROUND

Among the various subcellular localizations, the mitochondrion is characterized by a peculiar structure, comprised within two membranes. This organelle varies in size and shape and is organized in a network whose morphology is determined by the metabolic needs of the cell [1-3]. Mitochondria also contain multiple copies of a double-stranded circular genome (i.e. mitochondrial DNA, mtDNA) that encodes only 13 of the estimated 1,100 to 1,900 proteins of the organelle's proteome, with the remainder being encoded by nuclear DNA [4-6]. The mitochondrial transcriptome also comprises 16S ribosomal RNA molecules, transfer and small noncoding antisense mtRNA molecules.

In regard to their evolutive origin, mitochondria bear resemblances to bacteria, with features such as the presence of cardiolipin, N-formylated peptides, and hypomethylated CpG motifs that elicit pro-inflammatory responses upon their recognition by cells from the innate immune system [7]. Genomic studies also indicated that mtDNA shares similarities with the genome of Rickettsia prowazekii, a modern-era bacterium responsible for typhus [8]. This observation supports the theory suggesting that mitochondria are derived from the endosymbiosis between an α-proteobacterium and a primitive eukaryotic cell.

Despite their cytosolic nature, whole mitochondria and/or mitochondrial components may be released into the extracellular milieu in conditions of necrosis or tissue damage [9-12] or the activation of various cell lines [13-20]. Furthermore, cells may release intact and functional mitochondria that could be recaptured, allowing the recipient cell to rescue its damaged mitochondrial network [21,22]. Conversely, damaged mitochondria may be jettisoned by cells to preserve their functions [23,24]. The release of biomolecules (e.g. cytochrome c, mtDNA, ATP, reactive oxygen species, etc.) from the mitochondria, into the cytosol or the extracellular milieu, is also recognized as mitochondrial damage associated molecular patterns (mtDAMPs) skewing innate immunity toward a pro-inflammatory response [25].

Mitochondrial antigens may also be targeted by the adaptive immune system as indicated by the presence of a humoral response, comprised of various types of anti-mitochondrial autoantibodies (AMA) [26-29], in various inflammatory and autoimmune conditions. Although mitochondrial antigens were detected in association with both the major histocompatibility complex (MHC) molecules-I and -II [30-34], the precise pathophysiological pathway leading to the production of AMA is still unclear. In addition, the mitochondrial antigens targeted by several AMA remain to be characterized [28].

Systemic autoimmune rheumatic diseases (SARDs) include several diseases and disorders such as mixed connective tissue disease (MCTD), rheumatoid arthritis (RA), systemic lupus erythematosus (SLE), Sjögren's syndrome (SjS), systemic sclerosis (SSc), the group of the idiopathic inflammatory myositis (previously polymyositis (PM), and dermatomyositis (DM)), mixed connective tissue disease (MCTD) and the overlap and undifferentiated connective tissue diseases. SLE is a complex autoimmune disease in which the immune system generates autoantibodies recognizing self-epitopes. Antibodies directed against DNA and nuclear components are hallmarks of SLE [35-37]. Nevertheless, the appearance of antibodies directed against phospholipids and phospholipid-binding proteins (broadly referred to as antiphospholipid antibodies [aPL], as well as ribonucleoproteins have also been strongly associated with SLE. Furthermore, aPL are also present in patients with antiphospholipid syndrome, a syndrome which can also be diagnosed in patients with SLE (i.e., secondary APS). To date, there is a considerable obstacle in the diagnosis and classification of SARDs patients. In SLE, for example, certain existing diagnostic measures such as the American College of Rheumatology (ACR) criteria, Systemic Lupus International Collaborating Clinics (SLICC), European League Against Rheumatism (EULAR), and Systemic Lupus Erythematosus Disease Activity Index (SLEDAI) lack sensitivity and specificity. New measures for diagnosing these diseases are therefore needed.

Neutrophils and platelets are known sources of extracellular mitochondria in SLE [14,15,17,20,38,39]. Thus, antibodies directed against mitochondrial components may contribute to the disease pathogenesis through the formation of circulating antibody-antigen scaffolds called immune complexes. While the presence of antibodies against the mitochondrial phospholipid cardiolipin is well recognized, studies have also revealed that antibodies target mtDNA and mtRNA in SLE [27-29]. The 60-kDa heat-shock protein (HSP60), a chaperonin implicated in mitochondrial protein import, is encoded by mRNA from the nucleus. Although both healthy individuals and SLE patients present similar levels of antibodies against HSP60, the presence of these autoantibodies, in combination with increased antiphospholipids, is associated with arterial thrombosis [40,41].

Studies have revealed that antibodies could target mitochondria in SLE. However, the mitochondrial epitopes expressed on the outer membrane remains to be identified [26,27]. Herein, we aim to characterize the antigenic protein repertoire recognized by anti-mitochondrial antibodies in SLE patients.

### SUMMARY

In some embodiments described herein, is a method for identifying a subject having systemic lupus erythematosus (SLE) by detecting whether mitochondrial autoantibodies specific to one or more mitochondrial autoantigenic polypeptide are present; wherein said method comprises:
a. contacting a biological sample from a subject with one or more mitochondrial autoantigenic polypeptide to form a complex between each respective autoantibody and mitochondrial autoantigenic polypeptide;
b. detecting each complex between each respective autoantibody and mitochondrial autoantigenic polypeptide;
c. optionally, measuring the levels of autoantibody specific to one or more mitochondrial autoantigenic polypeptide; and
d. identifying the subject having SLE when one or more autoantibody specific to the one or more mitochondrial autoantigenic polypeptide relative to a control are present;
wherein said one or more mitochondrial autoantigenic polypeptide is or comprises mitofusin-1 (Mfn-1) or C1qBP.

In some embodiments described herein, is a method for the treatment of systemic lupus erythematosus (SLE) in an SLE subject in need thereof, wherein said method comprises:
a. identifying an SLE subject by detecting whether mitochondrial autoantibodies specific to one or more mitochondrial autoantigenic polypeptide are present by a method which comprises:
b. contacting a biological sample from a human subject suspected of suffering from SLE with one or more mitochondrial autoantigenic polypeptide to form a complex between each respective autoantibody and mitochondrial autoantigenic polypeptide;
c. detecting each complex between each respective autoantibody and mitochondrial autoantigenic polypeptide;
d. optionally, measuring the levels of autoantibody specific to one or more mitochondrial autoantigenic polypeptide;
e. identifying the SLE subject when one or more autoantibody specific to the one or more mitochondrial autoantigenic polypeptide relative to a control are present; and
f. when the SLE subject is identified, treating the SLE subject with anti-SLE therapy; wherein said one or more mitochondrial autoantigenic polypeptide is or comprises mitofusin-1 (Mfn-1) or C1qBP.

In some embodiments described herein, is a method for diagnosing or determining a progression of systemic lupus erythematosus (SLE) in a subject, said method comprising:
a. providing a biological sample from the subject;
b. detecting one or more autoantibody levels specific to one or more mitochondrial autoantigenic polypeptide; and
c. diagnosing the subject as having SLE or determining the progression of SLE by observing significantly increased autoantibody levels of specific to the one or more mitochondrial autoantigenic polypeptide as compared to a subject not having SLE;
wherein said one or more mitochondrial autoantigenic polypeptide is or comprises mitofusin-1 (Mfn-1) or C1qBP.

In some embodiments described herein, is a method of detecting one or more autoantibodies specific to one or more mitochondrial autoantigenic polypeptide in a biological sample from a human subject suspected of having systemic lupus erythematosus (SLE); said method comprising:
a. contacting the biological sample from the human subject with one or more mitochondrial autoantigenic polypeptide to form a complex between each respective autoantibody and mitochondrial autoantigenic polypeptide;
b. detecting each complex between each respective autoantibody and mitochondrial autoantigenic polypeptide; and
c. optionally, measuring the levels of autoantibody specific to one or more mitochondrial autoantigenic polypeptide;
wherein said one or more mitochondrial autoantigenic polypeptide is or comprises mitofusin-1 (Mfn-1) or C1qBP.

In some embodiments described herein, is a kit for use in diagnosing or determining the progression of systemic lupus erythematosus (SLE) in a subject, said kit comprising one or more reagents for detecting autoantibodies specific to one or more mitochondrial autoantigenic polypeptides in a biological sample from the subject, wherein said one or more mitochondrial autoantigenic polypeptide is or comprises mitofusin-1 (Mfn-1) or C1qBP.

In some embodiments described herein, is a method of producing a complex comprising one or more mitochondrial autoantigenic polypeptides and one or more autoantibodies specific to the respective one or more mitochondrial autoantigenic polypeptides; wherein said method comprises:
a. contacting a biological sample from a subject comprising one or more autoantibodies, with one or more mitochondrial autoantigenic polypeptides, wherein the subject has or is suspected to have systemic lupus erythematosus (SLE); and
b. detecting each complex between each respective autoantibody and mitochondrial autoantigenic polypeptide;
wherein said one or more mitochondrial autoantigenic polypeptide is or comprises mitofusin-1 (Mfn-1) or C1qBP.

In some embodiments described herein, is a complex for use in the identification, diagnosis, or determination of progression of systemic lupus erythematosus (SLE) in a subject, wherein the complex comprises or is formed by one or more mitochondrial autoantigenic polypeptides and one or more autoantibodies specific to the respective one or more mitochondrial autoantigenic polypeptides, wherein said one or more mitochondrial autoantigenic polypeptide is or comprises mitofusin-1 (Mfn-1) or C1qBP.

In some embodiments described herein, is a complex for use as a research tool in the detection of one or more autoantibodies specific to the one or more mitochondrial autoantigenic polypeptides in a biological sample, wherein the complex comprises one or more mitochondrial autoantigenic polypeptides and one or more autoantibodies specific to the respective one or more mitochondrial autoantigenic polypeptides, wherein said one or more mitochondrial autoantigenic polypeptide is or comprises mitofusin-1 (Mfn-1) or C1qBP.

In some embodiments described herein, is a kit for use in the detection of autoantibodies in a biological sample, wherein the kit comprises autoantigens specific to the autoantibodies and reagents for the detection of the autoantibodies, wherein at least one of the autoantibodies are one or more autoantibodies specific to one or more mitochondrial autoantigenic polypeptides, and wherein at least one of the autoantigens are one or more mitochondrial autoantigenic polypeptides, wherein said one or more mitochondrial autoantigenic polypeptide is or comprises mitofusin-1 (Mfn-1) or C1qBP.

In some embodiments described herein, is a kit for use in the detection of one or more autoantibodies specific to a respective one or more mitochondrial autoantigenic polypeptides in a biological sample, wherein the kit comprises one or more mitochondrial autoantigenic polypeptides specific to the respective one or more autoantibodies and reagents for the detection of the one or more autoantibodies, wherein said one or more mitochondrial autoantigenic polypeptide is or comprises mitofusin-1 (Mfn-1) or C1qBP.

In some embodiments described herein, is a method of manufacturing the kit defined herein, said method comprising:
a. immobilizing the one or more mitochondrial autoantigenic polypeptides; and
b. providing reagents for the detection of autoantibodies specific to the respective said one or more mitochondrial autoantigenic polypeptides defined herein.

In some embodiments described herein, is a method of manufacturing the kit defined herein, said method comprising:
a. immobilizing the autoantigens; and
b. providing reagents for the detection of autoantibodies specific to the respective autoantigens.

In some embodiments described herein, is a method for producing or modifying a test for detecting systemic lupus erythematosus, the method comprising adding or integrating into said test quantifying a panel of mitochondrial autoantibodies, the panel comprising mitochondrial autoantibodies specific to one or more mitochondrial autoantigenic polypeptide, wherein said one or more mitochondrial autoantigenic polypeptide is or comprises mitofusin-1 (Mfn-1) or C1qBP.

### BRIEF DESCRIPTION OF THE DRAWINGS

In the appended drawings:

FIG. 1 shows the workflow used for the detection of mitochondrial antigens targeted by anti-mitochondrial antibodies in SLE. Top panel: Freshly isolated intact mitochondria were incubated with pooled sera from either 10 healthy donors or 10 SLE patients with high levels of anti-whole mitochondrial IgG (AwMA IgG). Mitochondria incubated with AwMA were then lysed and AwMA-IgG isolated with Dynabeads™. Bottom panel: An irrelevant monoclonal IgG targeting FcγRIIa—a protein absent from mitochondria, is bound to Dynabeads™ and incubated with mitochondrial lysates in order to identify non-specific binding of mtAgs. For each approach, samples were acquired in triplicate and mtAgs were identified by mass-spectrometry (MS).

FIG. 2 shows mitochondrial sublocalizations of the mitochondrial proteins identified. While some of the proteins enriched in SLE patients (i.e., 51 [29.48%]) were not assigned to a specific sublocalization, 63 (36.42%) proteins were located in the inner membrane (MIM), 41 (23.70%) hits were identified in the mitochondrial matrix (MM), 3 (1.73%) in the intermembrane space (IMS), and 21 (12.14%) at the surface of the outer membrane (MOM).

FIG. 3 shows the 173 mitochondrial proteins identified as targeted by autoantibodies in SLE and their sublocalization.

FIG. 4 shows high-confidence protein interaction network of proteins enriched in SLE patients. A protein interacting with the globular head of the C1q component of the complement (i.e., C1qBP) is also associated with the serin protease inhibitors (serpin) superfamily (arrow). Mitofusin 1 (Mfn1, arrowhead) is associated with the pyruvate dehydrogenase complex subunit E1 (PDC-E1) protein network.

FIG. 5 shows the subcellular locations, processes and functions of the proteins targeted by AMA in SLE.

FIG. 6 shows identified proteins, plotted in the order of increasing ratio. A ratio>1 suggests enrichment in SLE patients relative to healthy individuals.

FIG. 7 shows the immunoreactivity against mitochondrial proteins assessed by direct ELISA. The receptor for the complement component C1q (C1qBP) is a protein stored within the mitochondrion and subsequently dispatched to the cell surface and/or released into the extracellular space. SLE patients display increased levels of IgGs targeting C1qBP, compared with healthy individuals (p=0.049). Mitofusin 1 (Mfn1) is a protein expressed at the surface of the mitochondrion and is responsible for the fusion of mitochondrial outer membranes; anti-Mfn-1-IgG are significantly increased in patients with SLE, compared with healthy donors (p=0.0044). Furthermore, anti-C1qBP IgG and anti-Mfn-1 IgG antibodies were not significantly elevated in antiphospholipid syndrome (APS) or primary biliary cirrhosis (PBC) patients compared to healthy individuals. Healthy: n=30, SLE: n=87, APS: n=27, and PBC: n=12. Autoantibodies against mitochondrial proteins implicated in the urea cycle (i.e., OTC, NAGS, CPS1), OxPhos (i.e., ATP5F1B, ETFA) and mitochondrial metabolism (i.e., GOT2, ALDH2) were assessed in serum samples from the SARD-BDB (Anti-OTC-IgG: Healthy n=30, SLE: n=87. Other autoantibodies: Healthy n=10, SLE: n=30). Data are mean optical densities read at 405 nm (OD405 nm) standard deviation. Data are mean optical densities read at 405 nm (OD405 nm)±standard deviation. Wilcoxon-Mann-Whitney test. Ns (not significant): p>0.05; *: p≤0.05; **: p<0.01.

FIG. 8 shows the indirect immunofluorescence (IIF) staining of HEp-2 cells represented by z-stack projections of 1 μm total thickness. FIG. 8A, HEp-2 cells were stained with an isotype-matched irrelevant antibody (Control a [Neg]), or commercial antibodies (5 μg/ml each) specific to either mitofusin 1 (anti-Mfn-1) or complement component C1q binding protein (anti-C1qBP). While anti-Mfn-1 displayed a reticular staining of the cytoplasm typical of antimitochondrial antibodies (Mito; arrowheads in anti-Mfn-1 image), anti-C1qBP displayed speckled staining of the nucleus and the cytoplasm (S; arrows in anti-C1qBP image). FIG. 8B, Top panels show HEp-2 cells stained with fluorescein isothiocyanate-labeled anti-human IgG secondary antibody. Human serum-based negative control (Control b [Neg]) and diluted serum (1:80) from healthy donors (negative for anti-whole mitochondrial antibodies [AwMAs]) (Healthy [Neg]), displayed no signal. Serum from primary biliary cirrhosis (PBC) patients showed the typical mitochondrial-associated cytoplasmic reticular staining pattern displayed by antimitochondrial antibodies (AMAs). Bottom panels show routine clinical staining of HEp-2 cells with AwMA-positive SLE serum, which allowed for the observation of various nuclear fluorescence patterns (i.e., speckled, homogenous [H], dots). No samples qualified as positive for AMAs. However, confocal microscopy revealed that some samples Q18 may display cytoplasmic patterns resembling those seen in PBC. Insets show higher-magnification views of portions of the main images. Bars=10 μm.

FIG. 9 shows the qualitative associations between anti-C1qBP, anti-Mfn1, and autoantibodies routinely assessed in SLE. Data are median±interquartile range (IQR). Associations presented, herein, for anticardiolipins or anti-β₂-GPI were tested for IgGs.

FIG. 10 shows the correlations between anti-C1qBP, anti-Mfn1, and various continuous variables in SLE patients. Correlations presented, herein, for anticardiolipins or anti-β₂-GPI were tested for IgGs.

FIG. 11 shows the sociodemographic and clinical characteristics of the SLE patients included in the SARD biobank and data repository (SARD-BDB).

FIG. 12 shows the SLE disease characteristics expressed by the patients included in the SARD-BDB.

FIG. 13 shows the continuous variables acquired in blood samples acquired from SLE patients drawn at the time of their inclusion in the SARD-BDB.

### DETAILED DESCRIPTION

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about”, when used herein, indicates that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9, and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “protein” or “polypeptide” or “peptide” means any peptide-linked chain of amino acids, which may or may not comprise any type of modification (e.g., chemical or post-translational modifications such as acetylation, phosphorylation, glycosylation, sulfatation, sumoylation, prenylation, ubiquitination, etc.).

As used herein, “autoantibody” refers to an antibody produced by the immune system of a subject and that is directed to a self-antigen.

As used herein “mitochondrial autoantibodies specific to one or more mitochondrial autoantigenic polypeptide” refers to an autoantibody that specifically binds the target autoantigenic mitochondrial polypeptide or a fragment thereof.

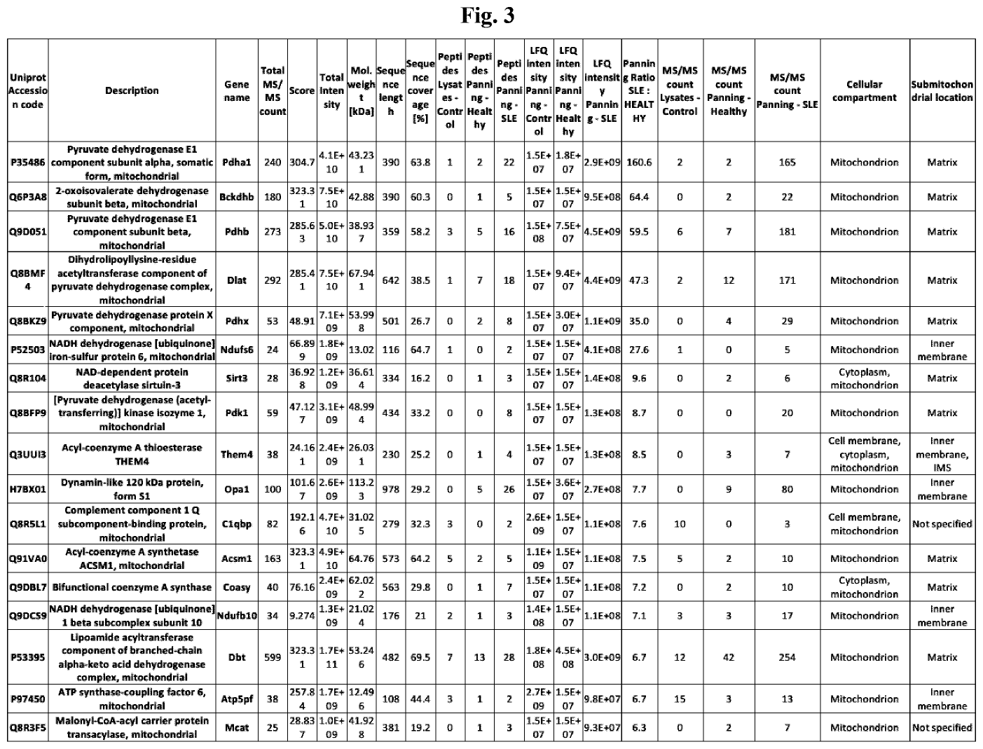

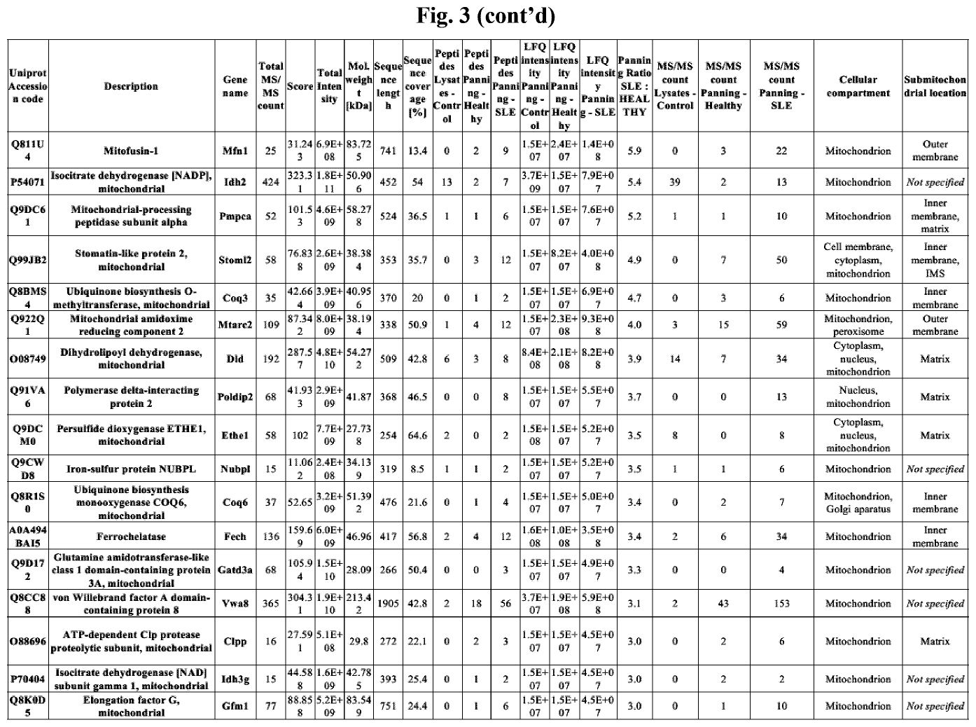

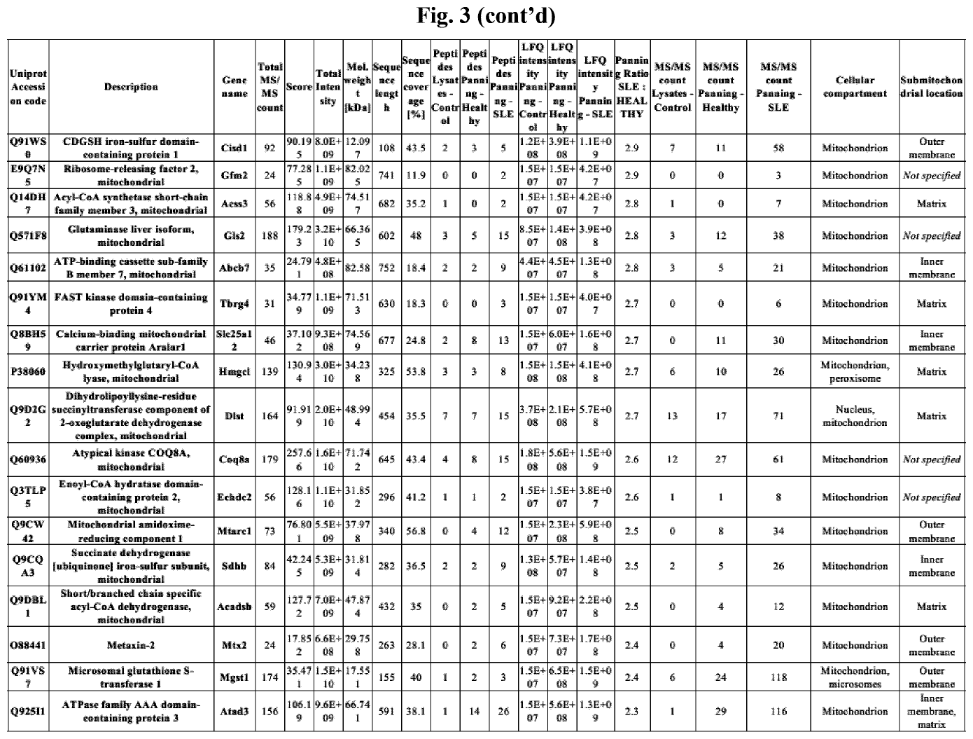

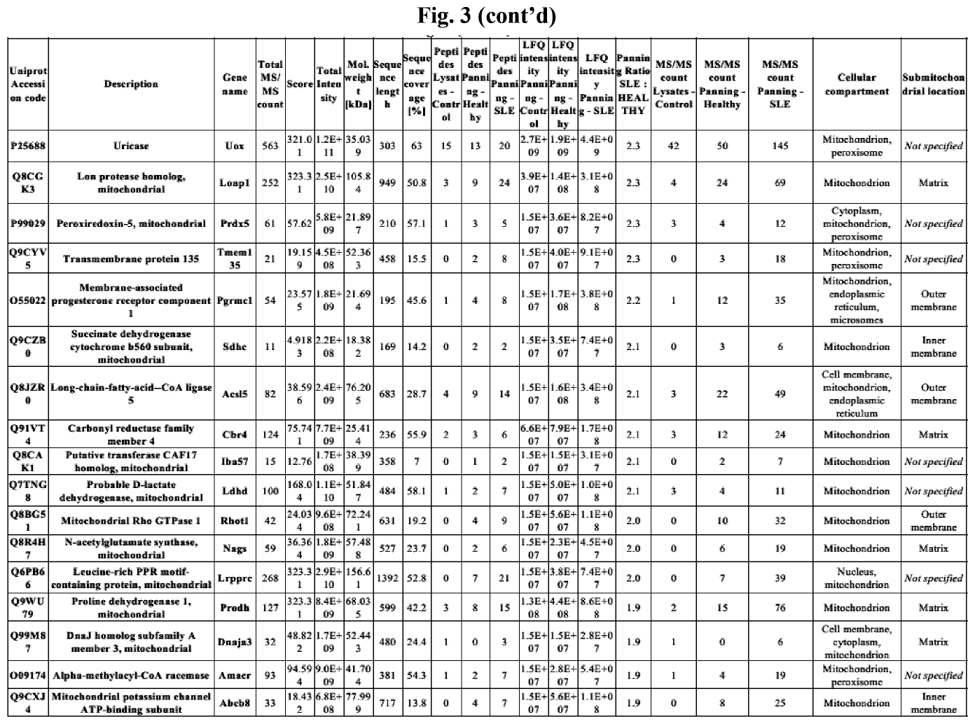

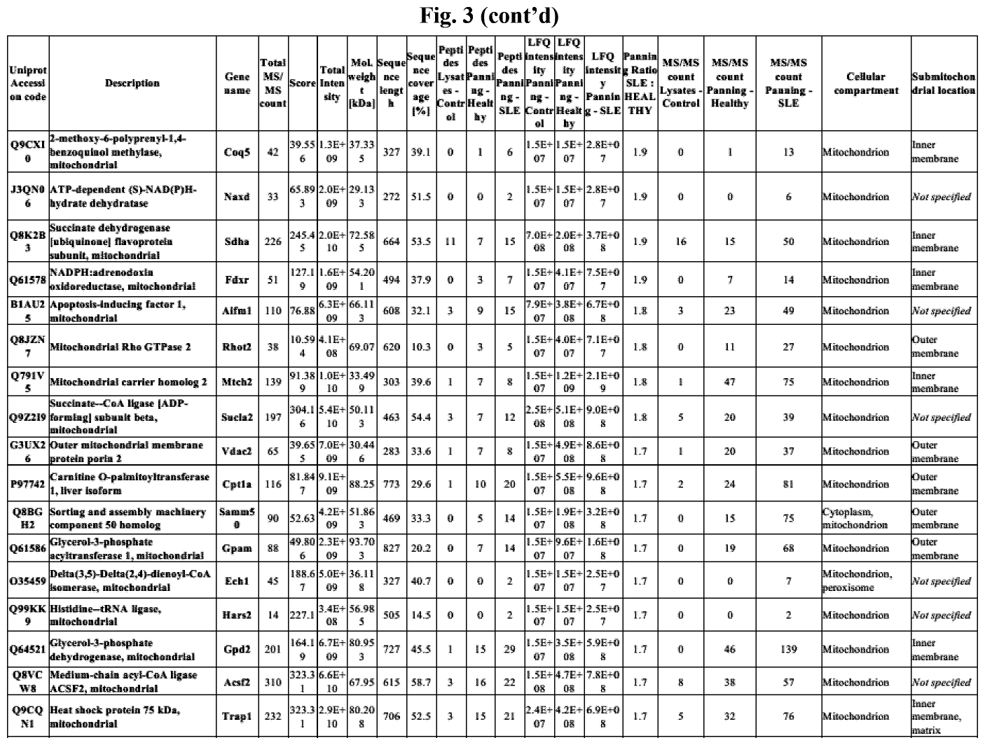

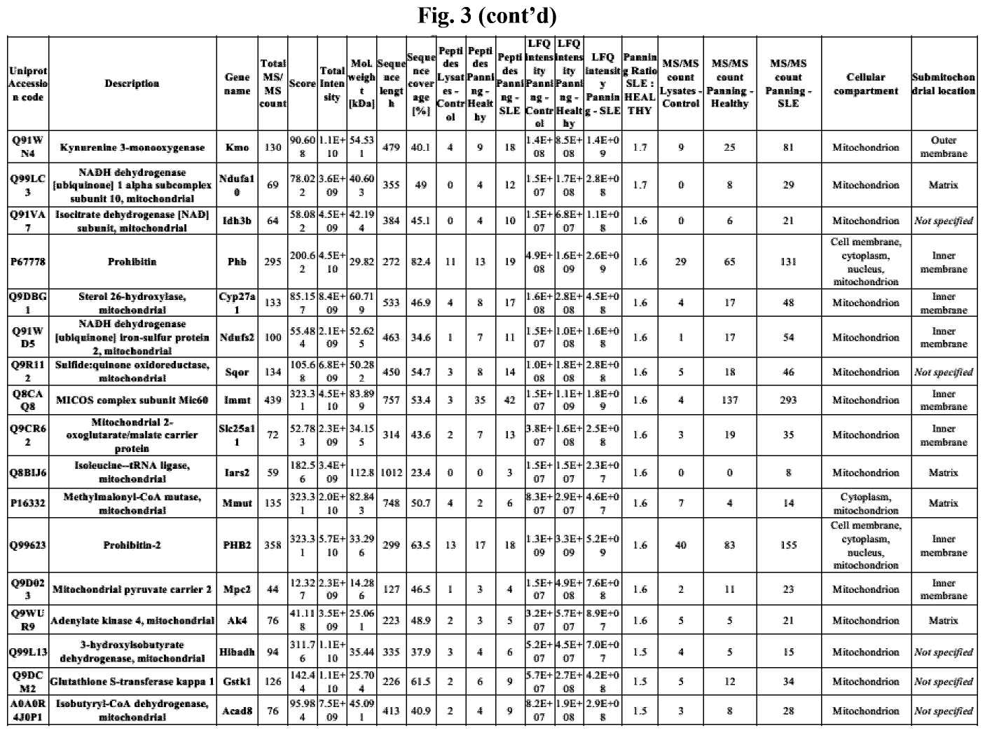

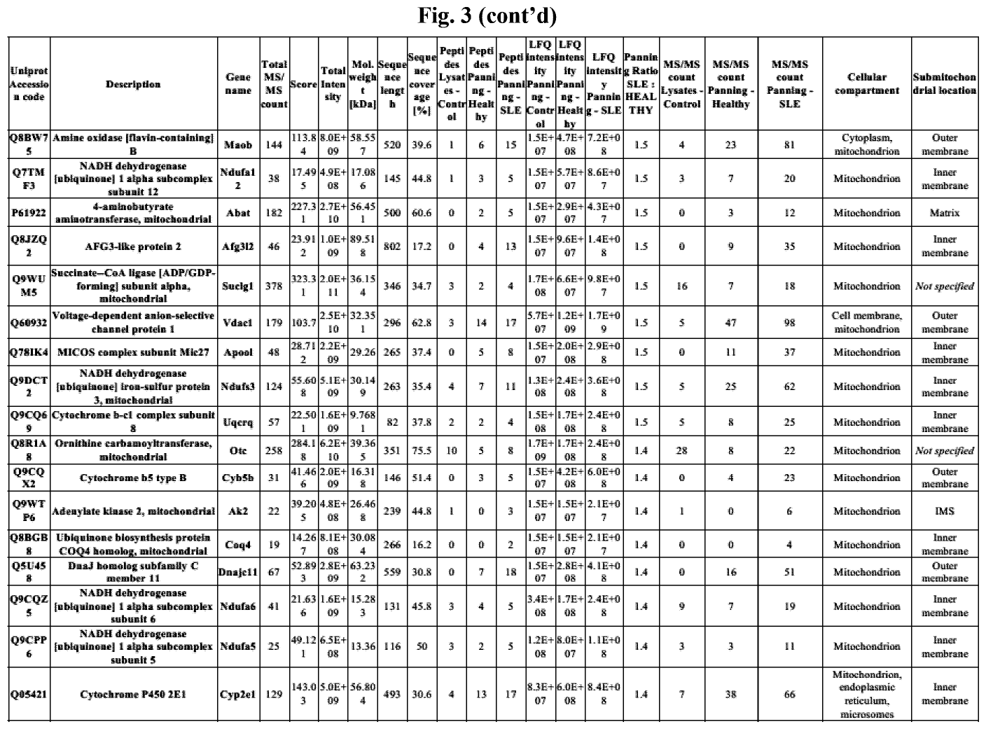

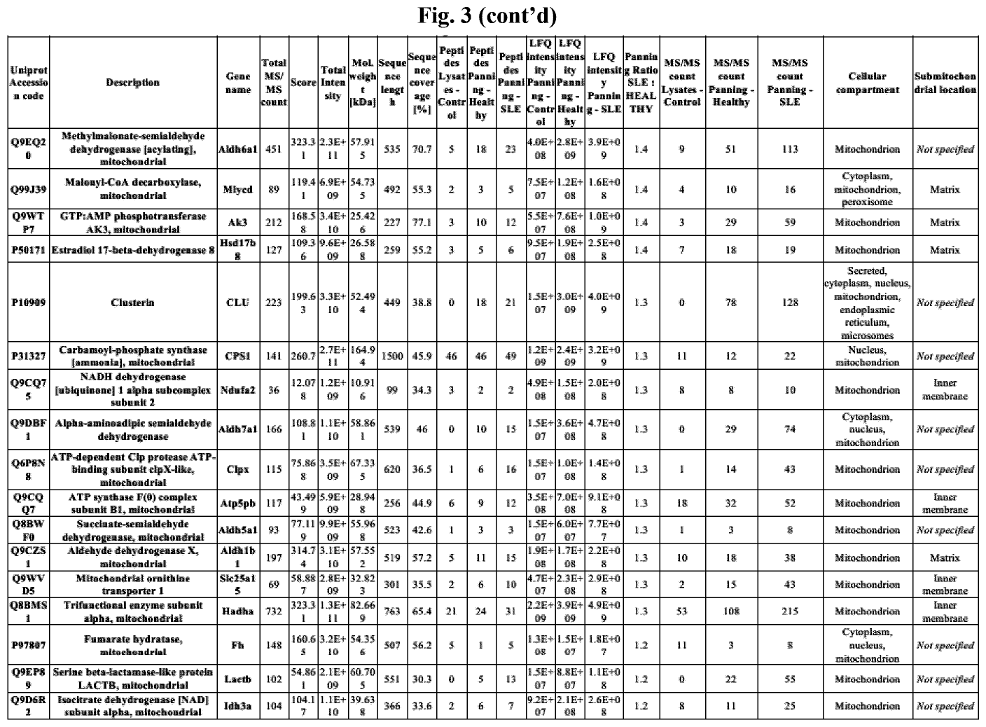

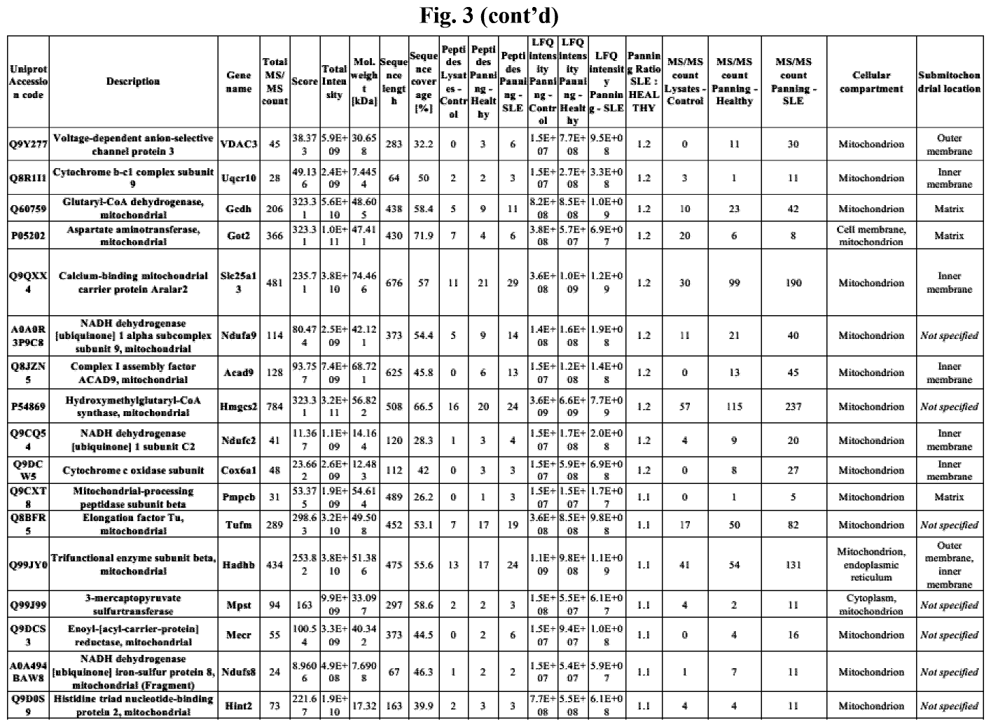

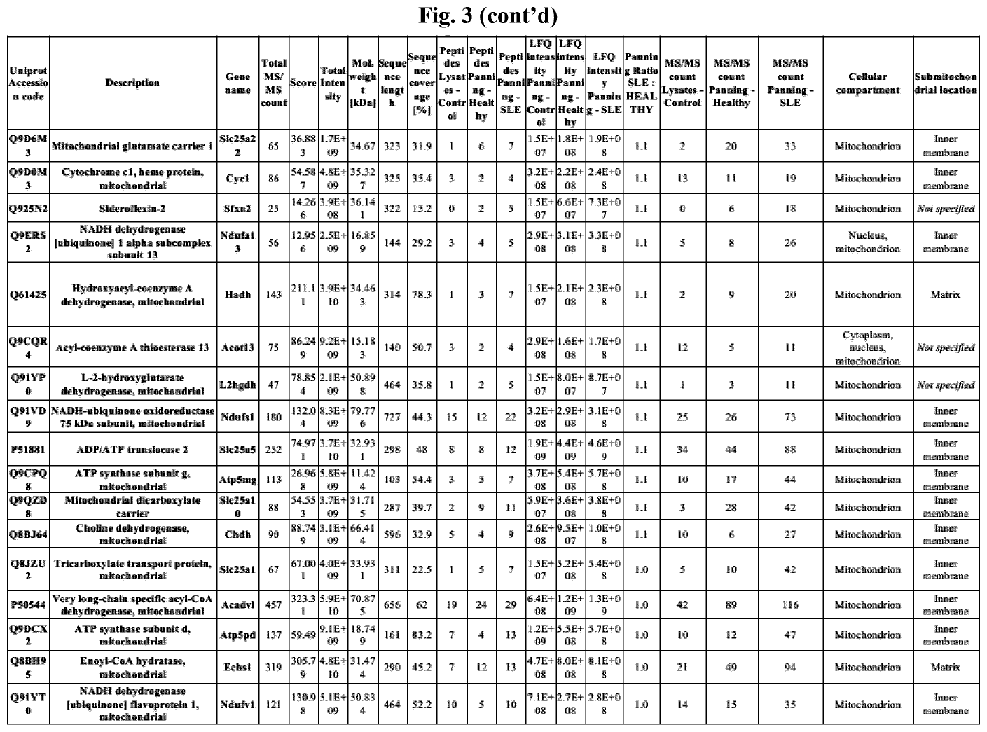

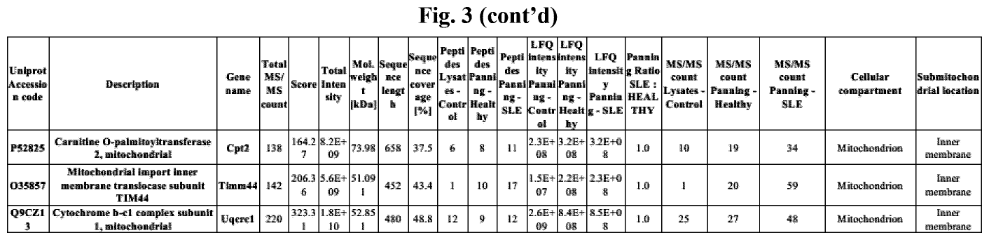

As used herein, “autoantigenic mitochondrial polypeptide” refers to a polypeptide that is at any point located in or bound to the mitochondria, including but not limited to polypeptides synthesized in the mitochondria or derived from mitochondrial DNA or RNA. A mitochondrial polypeptide may be localized in any compartment of the mitochondria, including but not limited to the mitochondrial outer membrane (MOM), inner membrane (MIM), intermembrane space (IMS), crystal membranes, intracrystal space, and matrix (MM). Mitochondrial outer membrane (MOM) proteins may include but are not limited to proteins that are constitutively or transiently expressed on the surface of the mitochondria, polypeptides that are bound to the surface of the mitochondria, or polypeptide that can bind to the surface of the mitochondria. Autoantigenic mitochondrial polypeptides include those recited in FIG. 3. In one aspect, the autoantigenic mitochondrial polypeptide is a heterologous polypeptide. In this context “heterologous” refers to a polypeptide that is from a different subject, organisms, or species.

As used herein, “systemic autoimmune rheumatic diseases (SARDs)” refers to an autoimmune disease or disorder that involves or is mediated by autoantibodies, including but not limited to rheumatoid arthritis (RA), systemic lupus erythematosus (SLE), Sjögren's syndrome (SjS), systemic sclerosis (SSc), the group of the idiopathic inflammatory myositis (IIM) (previously polymyositis [PM] and dermatomyositis [DM]), mixed connective tissue disease (MCTD) and the overlap and undifferentiated connective tissue diseases, or related diseases thereof.

As used herein, “subject” refers to a mammal, including but not limited to a human, mouse, rat, or rabbit. The subject may be a human patient, such as a patient having or suspected of having a SARD. The subject may also be a “healthy”, “reference” or “control” subject, which may not suffer from a SARD or does not present symptoms associated with a SARD and may be a healthy subject, which may be used as a control or reference in the determination of the presence, diagnosis, prognosis, classification, or progression of a SARD. Symptoms associated with a SARD may include but are not limited to a rash (such as a malar or butterfly rash), dry skin, shortness of breath, dermatitis, muscle or joint pain, swelling, or stiffness, inflammation, muscle weakness, fever, chest pain, hair loss, sun or light sensitivity, kidney malfunction, sores, blood clotting, fatigue, anemia, low blood cell counts, low white blood cells, low platelets, neurologic problem (such as seizures, strokes or psychosis), calcium deposits, diarrhea, constipation, Raynaud's phenomenon, and/or arrhythmia.

As used herein, “sample” or “biological sample” refers to any sample comprising or being tested for the presence of antibodies. Samples can include but are not limited to cellular extracts, extracellular fluid, fluid harvested from the body of a subject, culture media, blood, bone marrow, plasma, serum, biopsy, or any organ.

In one aspect described herein is a method for identifying, prognosing, classifying, or diagnosing a subject having an autoimmune disease comprising detecting one or more autoantibodies specific to one or more mitochondrial proteins. According to some aspects, the autoimmune disease includes but not limited to rheumatological diseases. According to some aspects, the rheumatological diseases include but are not limited to systemic autoimmune rheumatoid diseases (SARDs). According to some aspects, the method may further classify or determine the progression of the autoimmune disease.

In one aspect, described herein is a method for identifying a subject having a systemic autoimmune rheumatoid disease (SARD) in a biological sample from a subject comprising antibodies; wherein said method comprises providing one or more ligand specific to one or more mitochondrial proteins; contacting the biological sample from the subject with said one or more ligand specific to one or more mitochondrial proteins; detecting one or more autoantibodies specific to the one or more mitochondrial proteins; and identifying a patient having a SARD when one or more autoantibody specific to the one or more mitochondrial proteins is/are detected. The method described herein optionally includes measuring the levels of one or more autoantibodies specific to one or more mitochondrial proteins; and identifying a patient having a SARD when one or more autoantibody specific to the one or more mitochondrial proteins relative to a control present.

In one aspect, described herein is a method for diagnosing or determining the progression of a systemic autoimmune rheumatoid disease (SARD) in a subject, said method comprising providing a biological sample from the subject; detecting one or more autoantibody levels specific to one or more mitochondrial proteins; and diagnosing the subject as having the SARD or determining the progression of the SARD. The method optionally includes diagnosing the subject as having the SARD or determining the progression of the SARD by observing significantly increased autoantibody levels of specific to the one or more mitochondrial proteins as compared to a healthy subject or a subject not having SARD.

In one aspect, described herein is a method for identifying, prognosing, diagnosing, classifying, or determining a progression of a systemic autoimmune rheumatoid disease (SARD) in a subject, said method comprising: providing a biological sample from the subject; detecting autoantibodies specific to C1qBP and/or mitofusin 1 (Mfn1); optionally, measuring the levels autoantibodies specific to C1qBP and/or mitofusin 1 (Mfn1); and identifying, prognosing, diagnosing, classifying, or determining the progression of the subject having a SARD when one or more autoantibody specific to the one or more mitochondrial proteins relative to a control present.

In one aspect described herein, is a method for the treatment of a systemic autoimmune rheumatoid disease (SARD) in a SARD subject in need thereof; wherein said subject is identified as having a SARD by the methods described herein, and wherein the SARD subject is treated with anti-SARD therapy. Anti-SARD or anti-SLE therapy may include but is not limited to anti-inflammatory or non-steroidal anti-inflammatory drugs, corticosteroids, corticosteroid-sparing agents, analgesics, antimalarial drugs (such as hydroxychloroquine), immunosuppressants, biologics (such as belimumab, and rituximab), intravenous immunoglobulins, chemotherapies, disease-modifying antirheumatic drugs, sunscreens, transplantation, and/or surgery.

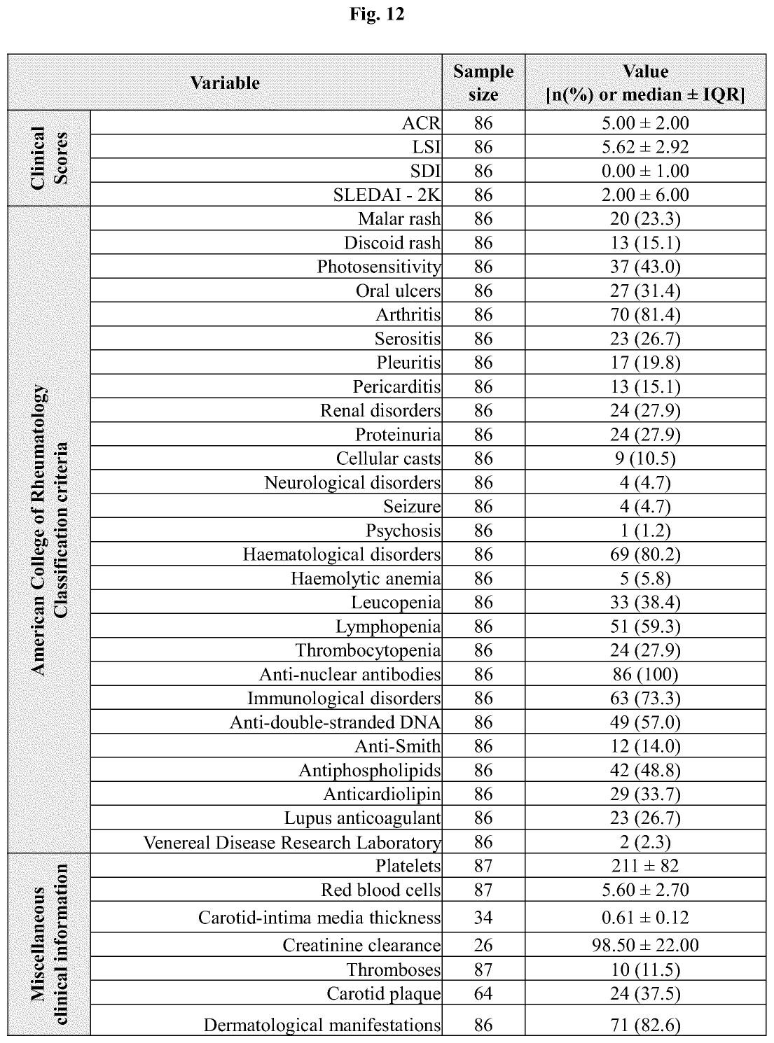

In some aspects, the methods described herein include identifying an SLE patient having a specific clinical manifestation (e.g., lupus nephritis, thrombosis). In some aspects, the method includes treating specific manifestations of SLE in a subject, such manifestations may include but are not limited to lupus nephritis and arterial/venous thrombosis or those shown in FIG. 12.

In one aspect, described herein is a method of detecting, screening, or panning of one or more antibodies or autoantibodies to one or more mitochondrial proteins.

In one aspect, described herein a kit comprising instructions to perform the methods described herein.

In one aspect, there is described a kit comprising one or more reagents for detecting autoantibodies specific to one or more mitochondrial proteins in a biological sample from the subject. The kit may further include a plate (such as a microplate), tube, or any suitable support, upon which a protein (such as an autoantigen) is or may be immobilized. Reagents for the kits described herein may include but are not limited to reagents commonly used in an immunoassay, such as in an enzyme-linked immunosorbent assay (ELISA). For example, the reagents may include a detecting antibody for the detection of autoantibodies bound to an immobilized autoantigen.

In one aspect, described herein is a kit for use in the diagnosis, prognosis, identification, classification, or determination of the progression of an autoimmune disease. According to some aspects, the autoimmune disease includes but not limited to rheumatological diseases. According to some aspects, the rheumatological diseases include but are not limited to systemic autoimmune rheumatoid diseases (SARDs).

In one aspect, described herein is a kit for use in the diagnosis, prognosis, identification, classification, or determination of progression of a SARD in a subject, said kit comprising one or more reagents for detecting autoantibodies specific to one or more mitochondrial proteins in a biological sample from the subject.

In one aspect, the one or more mitochondrial autoantigenic polypeptides and one or more autoantibodies specific to the respective one or more mitochondrial autoantigenic polypeptides (for example a heterologous polypeptides) can form a complex. The complex can also be isolated. The complex can be an immunocomplex.

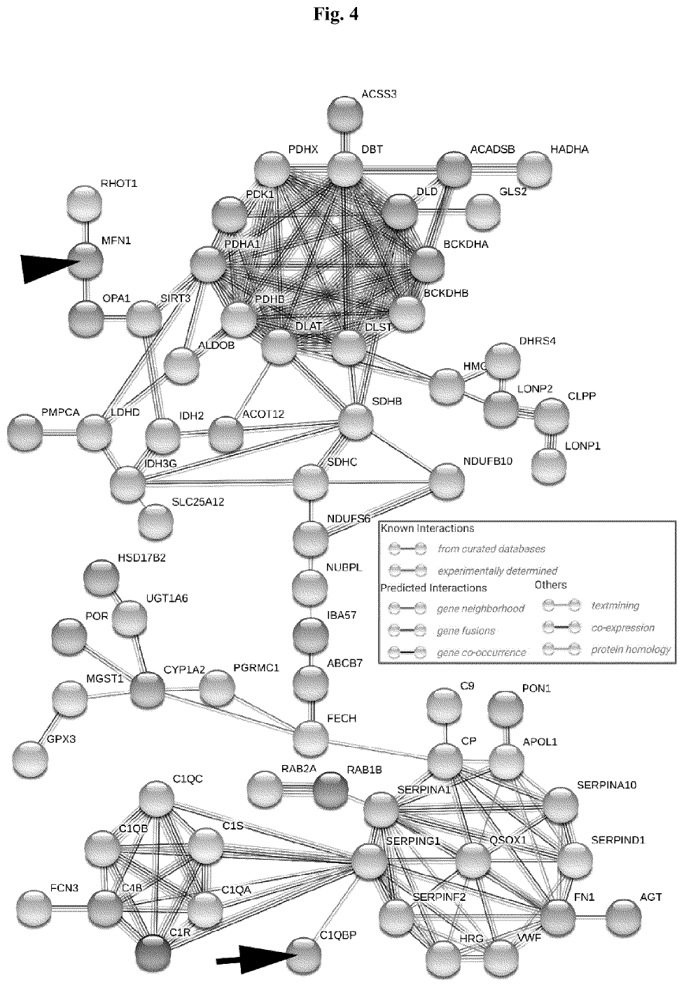

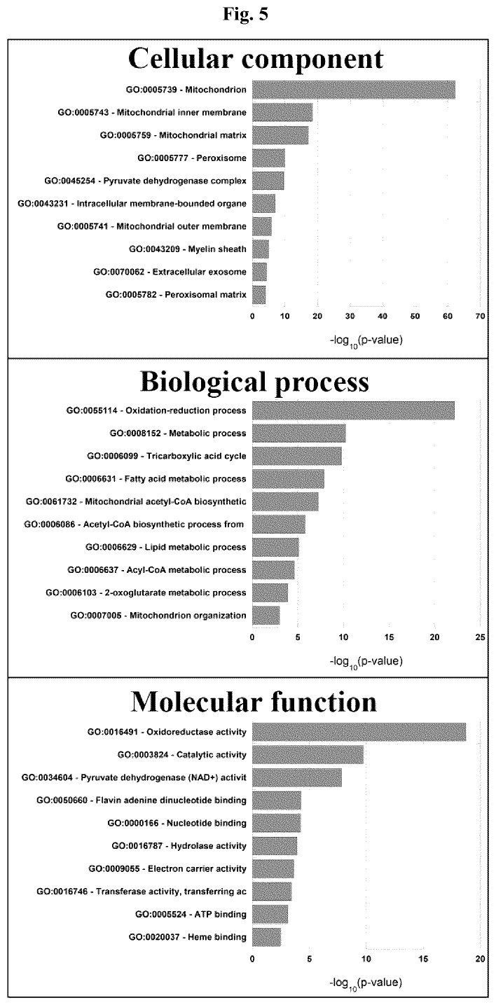

According to some aspects, the one or more mitochondrial proteins may be a mitochondrial outer membrane protein, a mitochondrial inner membrane protein, a mitochondrial matrix protein, a mitochondrial intermembrane space protein, a protein involved in the urea cycle, a protein involved in electron transfer and/or oxidative phosphorylation (OxPhos) pathways, a mitochondrial metabolism protein, or any combination thereof. The one or more mitochondrial proteins belonging to any networks listed in FIG. 4 and/or any of the pathways listed in FIG. 5. The one or more mitochondrial proteins may be one or more mitochondrial proteins listed in FIG. 3 or any combination thereof. For example, the one or more mitochondrial proteins may be pyruvate dehydrogenase E1 component subunit alpha, somatic form, mitochondrial, 2-oxoisovalerate dehydrogenase subunit beta, mitochondrial, pyruvate dehydrogenase E1 component subunit beta, mitochondrial, dihydrolipoyllysine-residue acetyltransferase component of pyruvate dehydrogenase complex, mitochondrial, pyruvate dehydrogenase protein X component, mitochondrial, NADH dehydrogenase [ubiquinone] iron-sulfur protein 6, mitochondrial, NAD-dependent protein deacetylase sirtuin-3, [pyruvate dehydrogenase (acetyl-transferring)] kinase isozyme 1, mitochondrial, acyl-coenzyme A thioesterase THEM4, dynamin-like 120 kDa protein, form Si, Complement component 1 Q subcomponent-binding protein, mitochondrial, acyl-coenzyme A synthetase ACSM1, mitochondrial, bifunctional coenzyme A synthase, NADH dehydrogenase [ubiquinone] 1 beta subcomplex subunit 10, lipoamide acyltransferase component of branched-chain alpha-keto acid dehydrogenase complex, mitochondrial, ATP synthase-coupling factor 6, mitochondrial, malonyl-CoA-acyl carrier protein transacylase, mitochondrial, mitofusin-1, isocitrate dehydrogenase [NADP], mitochondrial, mitochondrial-processing peptidase subunit alpha, stomatin-like protein 2, mitochondrial, ubiquinone biosynthesis O-methyltransferase, mitochondrial, mitochondrial amidoxime reducing component 2, dihydrolipoyl dehydrogenase, mitochondrial, polymerase delta-interacting protein 2, persulfide dioxygenase ETHE1, mitochondrial, iron-sulfur protein NUBPL, ubiquinone biosynthesis monooxygenase COQ6, mitochondrial, ferrochelatase, glutamine amidotransferase-like class 1 domain-containing protein 3A, mitochondrial, von Willebrand factor A domain-containing protein 8, ATP-dependent Clp protease proteolytic subunit, mitochondrial, isocitrate dehydrogenase [NAD] subunit gamma 1, mitochondrial, elongation factor G, mitochondrial, CDGSH iron-sulfur domain-containing protein 1, ribosome-releasing factor 2, mitochondrial, acyl-CoA synthetase short-chain family member 3, mitochondrial, glutaminase liver isoform, mitochondrial, ATP-binding cassette sub-family B member 7, mitochondrial, FAST kinase domain-containing protein 4, calcium-binding mitochondrial carrier protein Aralar1, hydroxymethylglutaryl-CoA lyase, mitochondrial, dihydrolipoyllysine-residue succinyltransferase component of 2-oxoglutarate dehydrogenase complex, mitochondrial, atypical kinase COQ8A, mitochondrial, enoyl-CoA hydratase domain-containing protein 2, mitochondrial, mitochondrial amidoxime-reducing component 1, succinate dehydrogenase [ubiquinone] iron-sulfur subunit, mitochondrial, short/branched chain specific acyl-CoA dehydrogenase, mitochondrial, metaxin-2, microsomal glutathione S-transferase 1, ATPase family AAA domain-containing protein 3, uricase, Lon protease homolog, mitochondrial, peroxiredoxin-5, mitochondrial, transmembrane protein 135, membrane-associated progesterone receptor component 1, succinate dehydrogenase cytochrome b560 subunit, mitochondrial, long-chain-fatty-acid-CoA ligase 5, carbonyl reductase family member 4, putative transferase CAF17 homolog, mitochondrial, probable D-lactate dehydrogenase, mitochondrial, mitochondrial Rho GTPase 1, N-acetylglutamate synthase, mitochondrial, leucine-rich PPR motif-containing protein, mitochondrial, proline dehydrogenase 1, mitochondrial, DnaJ homolog subfamily A member 3, mitochondrial, alpha-methylacyl-CoA racemase, mitochondrial potassium channel ATP-binding subunit, 2-methoxy-6-polyprenyl-1,4-benzoquinol methylase, mitochondrial, ATP-dependent (S)-NAD(P)H-hydrate dehydratase, succinate dehydrogenase [ubiquinone] flavoprotein subunit, mitochondrial, NADPH:adrenodoxin oxidoreductase, mitochondrial, apoptosis-inducing factor 1, mitochondrial, mitochondrial Rho GTPase 2, mitochondrial carrier homolog 2, succinate-CoA ligase [ADP-forming] subunit beta, mitochondrial, outer mitochondrial membrane protein porn 2, carnitine O-palmitoyltransferase 1, liver isoform, sorting and assembly machinery component 50 homolog, glycerol-3-phosphate acyltransferase 1, mitochondrial, delta(3,5)-delta(2,4)-dienoyl-CoA isomerase, mitochondrial, histidine-tRNA ligase, mitochondrial, glycerol-3-phosphate dehydrogenase, mitochondrial, medium-chain acyl-CoA ligase ACSF2, mitochondrial, heat shock protein 75 kDa, mitochondrial, kynurenine 3-monooxygenase, NADH dehydrogenase [ubiquinone] 1 alpha subcomplex subunit 10, mitochondrial, isocitrate dehydrogenase [NAD] subunit, mitochondrial, prohibitin, NADH dehydrogenase [ubiquinone] iron-sulfur protein 2, mitochondrial, sulfide:quinone oxidoreductase, mitochondrial, MICOS complex subunit Mic60, mitochondrial 2-oxoglutarate/malate carrier protein, isoleucine-tRNA ligase, mitochondrial, methylmalonyl-CoA mutase, mitochondrial, prohibitin-2, mitochondrial pyruvate carrier 2, adenylate kinase 4, mitochondrial, 3-hydroxyisobutyrate dehydrogenase, mitochondrial, glutathione S-transferase kappa 1, isobutyryl-CoA dehydrogenase, mitochondrial, amine oxidase [flavin-containing] B, NADH dehydrogenase [ubiquinone] 1 alpha subcomplex subunit 12, 4-aminobutyrate aminotransferase, mitochondrial, AFG3-like protein 2, succinate-CoA ligase [ADP/GDP-forming] subunit alpha, mitochondrial, voltage-dependent anion-selective channel protein 1, MICOS complex subunit Mic27, NADH dehydrogenase [ubiquinone] iron-sulfur protein 3, mitochondrial, cytochrome b-c1 complex subunit 8, ornithine carbamoyltransferase, mitochondrial, cytochrome b5 type B, adenylate kinase 2, mitochondrial, ubiquinone biosynthesis protein COQ4 homolog, mitochondrial, DnaJ homolog subfamily C member 11, NADH dehydrogenase [ubiquinone] 1 alpha subcomplex subunit 6, NADH dehydrogenase [ubiquinone] 1 alpha subcomplex subunit 5, cytochrome P450 2E1, methylmalonate-semialdehyde dehydrogenase [acylating], mitochondrial, malonyl-CoA decarboxylase, mitochondrial, GTP:AMP phosphotransferase AK3, mitochondrial, estradiol 17-beta-dehydrogenase 8, clusterin, carbamoyl-phosphate synthase [ammonia], mitochondrial, NADH dehydrogenase [ubiquinone] 1 alpha subcomplex subunit 2, alpha-aminoadipic semialdehyde dehydrogenase, ATP-dependent Clp protease ATP-binding subunit clpX-like, mitochondrial, ATP synthase F(0) complex subunit B1, mitochondrial, succinate-semialdehyde dehydrogenase, mitochondrial, aldehyde dehydrogenase X, mitochondrial, mitochondrial ornithine transporter 1, trifunctional enzyme subunit alpha, mitochondrial, fumarate hydratase, mitochondrial, serine beta-lactamase-like protein LACTB, mitochondrial, isocitrate dehydrogenase [NAD] subunit alpha, mitochondrial, voltage-dependent anion-selective channel protein 3, cytochrome b-c1 complex subunit 9, glutaryl-CoA dehydrogenase, mitochondrial, aspartate aminotransferase, mitochondrial, calcium-binding mitochondrial carrier protein Aralar2, NADH dehydrogenase [ubiquinone] 1 alpha subcomplex subunit 9, mitochondrial, complex I assembly factor ACAD9, mitochondrial, hydroxymethylglutaryl-CoA synthase, mitochondrial, NADH dehydrogenase [ubiquinone] 1 subunit C2, cytochrome c oxidase subunit, mitochondrial-processing peptidase subunit beta, elongation factor Tu, mitochondrial, trifunctional enzyme subunit beta, mitochondrial, 3-mercaptopyruvate sulfurtransferase, enoyl-[acyl-carrier-protein]reductase, mitochondrial, NADH dehydrogenase [ubiquinone] iron-sulfur protein 8, mitochondrial (fragment), histidine triad nucleotide-binding protein 2, mitochondrial, mitochondrial glutamate carrier 1, cytochrome c1, heme protein, mitochondrial, sideroflexin-2, NADH dehydrogenase [ubiquinone] 1 alpha subcomplex subunit 13, hydroxyacyl-coenzyme A dehydrogenase, mitochondrial, acyl-coenzyme A thioesterase 13, L-2-hydroxyglutarate dehydrogenase, mitochondrial, NADH-ubiquinone oxidoreductase 75 kDa subunit, mitochondrial, ADP/ATP translocase 2, ATP synthase subunit g, mitochondrial, mitochondrial dicarboxylate carrier, choline dehydrogenase, mitochondrial, tricarboxylate transport protein, mitochondrial, very long-chain specific acyl-CoA dehydrogenase, mitochondrial, ATP synthase subunit d, mitochondrial, enoyl-CoA hydratase, mitochondrial, NADH dehydrogenase [ubiquinone] flavoprotein 1, mitochondrial, carnitine O-palmitoyltransferase 2, mitochondrial, mitochondrial import inner membrane translocase subunit TIM44, cytochrome b-c1 complex subunit 1, mitochondrial, ornithine carbamoyltransferase (OTC), carbamoyl-phosphate synthase (CPS1), N-acetylglutamate synthase (NAGS), ATP synthase (ATP5F1B), flavoprotein subunit alpha (ETFA), aspartate aminotransferase (GOT2), aldehyde dehydrogenase (ALDH2), or any combination thereof.

According to some aspects, the one or more mitochondrial proteins is/are mitchondrial outer membrane proteins. According to some aspects, the one or more mitochondrial protein is/are C1qBP and/or mitofusin 1 (Mfn1).

According to some aspects, the SARD may be selected from but is not limited by the group consisting of mixed connective tissue disease (MCTD), rheumatoid arthritis (RA), systemic lupus erythematosus (SLE), Sjögren's syndrome (SjS), systemic sclerosis (SSc), polymyositis (PM), and dermatomyositis (DM).

According to some aspects, the SARD is systemic lupus erythematosus (SLE). In some aspects, systemic lupus erythematosus (SLE) is characterized as lupus, discoid lupus, drug-induced lupus, neonatal lupus, acute or subacute cutaneous lupus erythematosus, cutaneous lupus, and/or lupus nephritis.

In some aspects, SLE may be accompanied by a secondary syndrome or disorder, including but not limited to antiphospholipid syndrome.

According to some aspects, the detection of one or more autoantibody/autoantibodies is determined using commonly known methods for the detection of antibodies, including but not limited to an immunoassay. Immunoassays may include an enzyme-linked immunosorbent assay (ELISA), such as a direct binding or sandwich ELISA.

According to some aspects, the methods or kits described herein may further comprises a step of combining with levels of one or more autoantibodies known to be associated with the identification, diagnosis or progression of said SARD. Examples of these known autoantibodies include but are not limited to anti-nuclear antibodies, anti-double stranded DNA antibodies, lupus anticoagulant antibodies, antiphospholipid antibodies, anticardiolipin antibodies, anti-D2 Glycoprotein I antibodies, anti-ribonucleoprotein antibodies, anti-Smith (Sm) antibodies, anti-small nuclear ribonucleoprotein antibodies, anti-Ro (SS/A) antibodies, anti-La (SS/B) antibodies, anti-mitochondrial DNA, anti-whole mitochondria, anti-mitochondrial RNA antibodies, or any combination thereof.

According to some aspects, the methods or kits described herein further comprises a step of combining with one or more criteria selected from one or more known diagnostic or classification measures of systemic autoimmune rheumatoid disease (SARD). In some aspects, the one or more known diagnostic or classification measures may be but are not limited to the American College of Rheumatology (ACR) classification criteria, Systemic Lupus International Collaborating Clinics (SLICC) classification criteria, Systemic Lupus Erythematosus Disease Activity Index (SLEDAI), lupus severity index (LSI), Systemic Lupus International Collaborating Clinics/American College of Rheumatology (SLICC/ACR) Damage Index (SDI), or European League Against Rheumatism (EULAR) classification criteria.

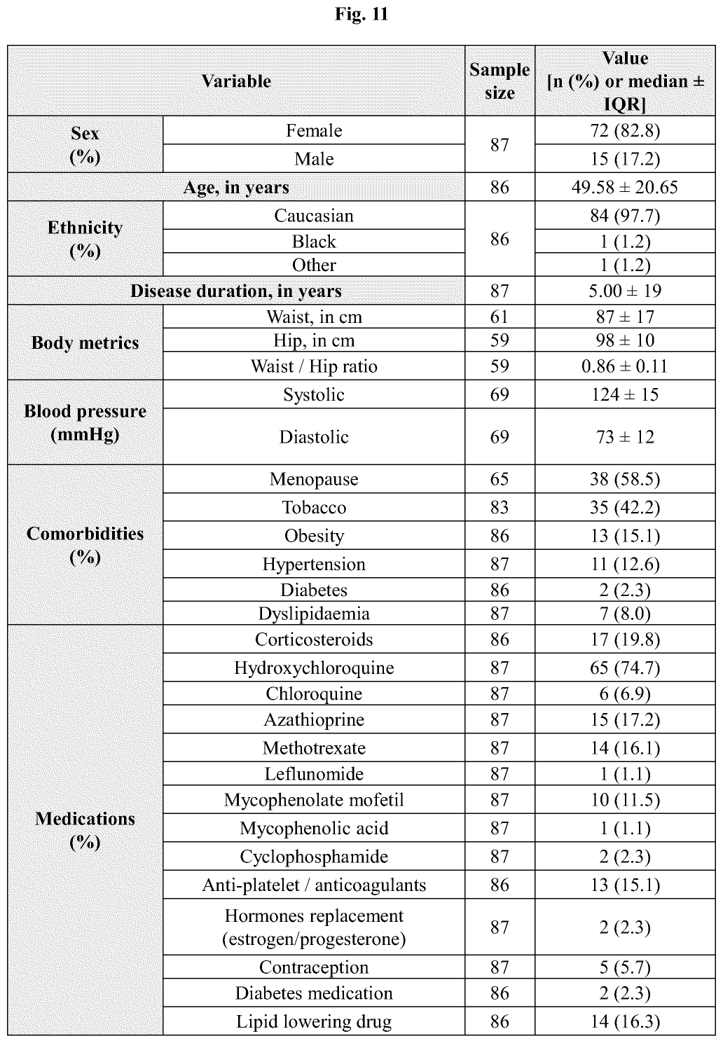

According to some aspects, the methods or kits described herein are combined with one or more criteria and/or measurements selected from FIGS. 11 and 12.

In one aspect, described herein is a method for producing or modifying a test for detecting systemic lupus erythematosus (SLE), the method comprising adding or integrating into said test quantifying a panel of mitochondrial autoantibodies, the panel comprising mitochondrial autoantibodies specific to one or more mitochondrial autoantigenic polypeptide. In some aspects, the one or more mitochondrial autoantigenic polypeptides comprise C1qBP and/or mitofusin 1 (Mfn1). In some aspects, the test for detecting SLE may include existing autoantibody tests for diagnosing SLE. Examples of these known autoantibodies include but are not limited to anti-nuclear antibodies, anti-double stranded DNA antibodies, lupus anticoagulant antibodies, antiphospholipid antibodies, anticardiolipin antibodies, anti-D2 Glycoprotein I antibodies, anti-ribonucleoprotein antibodies, anti-Smith (Sm) antibodies, anti-small nuclear ribonucleoprotein antibodies, anti-Ro (SS/A) antibodies, anti-La (SS/B) antibodies, anti-mitochondrial DNA, anti-whole mitochondria, anti-mitochondrial RNA antibodies, or any combination thereof. In some aspects, the test for detecting SLE may include one or more criteria selected from one or more known diagnostic or classification measures of systemic autoimmune rheumatoid disease (SARD). In some aspects, the one or more known diagnostic or classification measures may be but are not limited to the American College of Rheumatology (ACR) classification criteria, Systemic Lupus International Collaborating Clinics (SLICC) classification criteria, Systemic Lupus Erythematosus Disease Activity Index (SLEDAI), lupus severity index (LSI), Systemic Lupus International Collaborating Clinics/American College of Rheumatology (SLICC/ACR) Damage Index (SDI), or European League Against Rheumatism (EULAR) classification criteria.

In a further aspect, the following embodiments are provided:

Embodiment 1. A method for identifying a subject having a systemic autoimmune rheumatoid disease (SARD) by detecting whether mitochondrial autoantibodies specific to one or more mitochondrial autoantigenic polypeptide are present; wherein said method comprises:
a. contacting a biological sample from a subject with one or more mitochondrial autoantigenic polypeptide to form a complex between each respective autoantibody and mitochondrial autoantigenic polypeptide;
b. detecting each complex between each respective autoantibody and mitochondrial autoantigenic polypeptide;
c. optionally, measuring the levels of autoantibody specific to one or more mitochondrial autoantigenic polypeptide;
d. identifying the subject having a SARD when one or more autoantibody specific to the one or more mitochondrial autoantigenic polypeptide relative to a control are present.

Embodiment 2. A method for the treatment of a systemic autoimmune rheumatoid disease (SARD) in a SARD subject in need thereof, wherein said method comprises:
a. identifying a SARD subject by detecting whether mitochondrial autoantibodies specific to one or more mitochondrial autoantigenic polypeptide are present by a method which comprises:
i. contacting a biological sample from a human subject suspected of suffering from a SARD with one or more mitochondrial autoantigenic polypeptide to form a complex between each respective autoantibody and mitochondrial autoantigenic polypeptide;
ii. detecting each complex between each respective autoantibody and mitochondrial autoantigenic polypeptide;
iii. optionally, measuring the levels of autoantibody specific to one or more mitochondrial autoantigenic polypeptide; and
iv. identifying the SARD subject when one or more autoantibody specific to the one or more mitochondrial autoantigenic polypeptide relative to a control are present; and
b. when a SARD subject is identified, treating the SARD subject with anti-SARD therapy.

Embodiment 3. A method for diagnosing or determining a progression of a systemic autoimmune rheumatoid disease (SARD) in a subject, said method comprising:
a. providing a biological sample from the subject;
b. detecting one or more autoantibody levels specific to one or more mitochondrial autoantigenic polypeptide;
c. diagnosing the subject as having the SARD or determining the progression of the SARD by observing significantly increased autoantibody levels of specific to the one or more mitochondrial autoantigenic polypeptide as compared to a subject not having a systemic autoimmune rheumatoid disease (SARD).

Embodiment 4. The method of embodiment 1, 2 or 3, wherein the one or more mitochondrial autoantigenic polypeptide is/are:
a mitochondrial outer membrane polypeptide;
a mitochondrial inner membrane polypeptide;
a mitochondrial matrix polypeptide;
a mitochondrial intermembrane space polypeptide;
a protein involved in the urea cycle;
a protein involved in the electron transfer and/or oxidative phosphorylation (OxPhos) pathways;
a mitochondrial metabolism polypeptide;
a protein involved in any one of the networks listed in FIG. 4;
a protein involved in any one of the pathways listed in FIG. 5; or
any combination thereof.

Embodiment 5. The method of any one of embodiments 1 to 4, wherein the one or more autoantigenic polypeptide is/are mitochondrial outer membrane polypeptide (s).

Embodiment 6. The method of any one of embodiments 1 to 4, wherein the one or more autoantigenic polypeptide is/are the one or more autoantigenic polypeptide listed in FIG. 3 or any combination thereof.

Embodiment 7. The method of any one of embodiments 1 to 6, wherein two or more mitochondrial autoantigenic polypeptide are detected.

Embodiment 8. The method of any one of embodiments 1 to 6, wherein the one or more mitochondrial autoantigenic polypeptide is/are C1qBP and/or mitofusin 1 (Mfn1).

Embodiment 9. The method of any one of embodiments 1 to 8, wherein said system autoimmune rheumatoid disease is selected from the group consisting of mixed connective tissue disease (MCTD), rheumatoid arthritis (RA), systemic lupus erythematosus (SLE), Sjögren's syndrome (SjS), systemic sclerosis (SSc), polymyositis (PM), and dermatomyositis (DM).

Embodiment 10. The method of any one of embodiments 1 to 8, wherein said system autoimmune rheumatoid disease is systemic lupus erythematosus (SLE).

Embodiment 11. The method of embodiment 9 or 10, wherein the systemic lupus erythematosus (SLE) is accompanied by a secondary syndrome or disorder.

Embodiment 12. The method of embodiment 11, wherein the secondary syndrome or disorder is antiphospholipid syndrome.

Embodiment 13. The method of any one of embodiments 1 to 12, wherein the detecting of one or more autoantibody/autoantibodies is determined using an immunoassay.

Embodiment 14. The method of any one of embodiments 1 to 13, wherein the subject is a human.

Embodiment 15. The method of any one of embodiments 1 to 14, wherein the sample is blood, serum, or plasma.

Embodiment 16. The method of any one of embodiments 1 to 15, wherein the method further comprises a step of detecting or identifying the levels of one or more autoantibodies selected from the group consisting of anti-nuclear antibodies, anti-double stranded DNA antibodies, lupus anticoagulant antibodies, antiphospholipid antibodies, anticardiolipin antibodies, anti-D2 Glycoprotein I antibodies, anti-ribonucleoprotein antibodies, anti-small nuclear ribonucleoprotein antibodies, anti-Smith (Sm) anti-Ro (SS/A) antibodies, anti-La (SS/B) antibodies, anti-mitochondrial DNA, anti-whole mitochondria, anti-mitochondrial RNA antibodies, and any combination thereof.

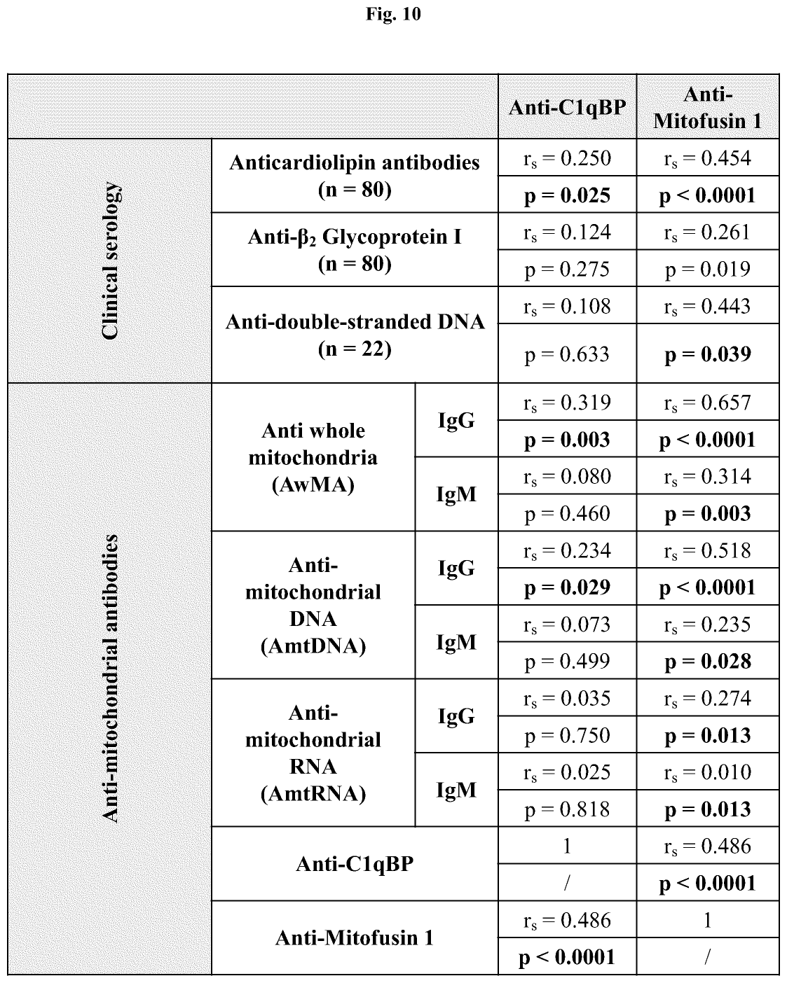

Embodiment 17. The method of any one of embodiments 1 to 16, wherein the method further comprises a step of determining whether one or more criteria selected from the group consisting of American College of Rheumatology (ACR) classification criteria, Systemic Lupus International Collaborating Clinics (SLICC) classification criteria, Systemic Lupus Erythematosus Disease Activity Index (SLEDAI), lupus severity index (LSI), Systemic Lupus International Collaborating Clinics/American College of Rheumatology (SLICC/ACR) Damage Index (SDI), European League Against Rheumatism (EULAR) classification criteria, and criteria selected from FIGS. 10 and/or 11 are present.

Embodiment 18. A method of detecting one or more autoantibodies specific to one or more mitochondrial autoantigenic polypeptide in a biological sample from a human subject suspected of having a systemic autoimmune rheumatoid disease (SARD); said method comprising:
a. contacting the biological sample from the human subject with one or more mitochondrial autoantigenic polypeptide to form a complex between each respective autoantibody and mitochondrial autoantigenic polypeptide;
b. detecting each complex between each respective autoantibody and mitochondrial autoantigenic polypeptide; and
c. optionally, measuring the levels of autoantibody specific to one or more mitochondrial autoantigenic polypeptide.

Embodiment 19. The method of embodiment 18, wherein one or more autoantibodies as defined in embodiment 16 are present in the human subject suspected of having the systemic autoimmune rheumatoid disease (SARD).

Embodiment 20. The method of embodiment 18 or 19, wherein one or more criteria as defined in embodiment 17 are present in the human subject suspected of having the systemic autoimmune rheumatoid disease (SARD).

Embodiment 21. The method of any one of embodiments 18 to 20, wherein two or more mitochondrial autoantigenic polypeptide are detected.

Embodiment 22. The method of any one of embodiments 18 to 21, further comprising one or more features as defined in any one of embodiments 4 to 17.

Embodiment 23. A kit for use in diagnosing or determining the progression of a systemic autoimmune rheumatoid disease (SARD) in a subject, said kit comprising one or more reagents for detecting autoantibodies specific to one or more mitochondrial autoantigenic polypeptides in a biological sample from the subject.

Embodiment 24. The kit of embodiment 23, wherein the one or more mitochondrial proteins is/are:
a mitochondrial outer membrane polypeptide;
a mitochondrial inner membrane polypeptide;
a mitochondrial matrix polypeptide;
a mitochondrial intermembrane space polypeptide;
a protein involved in the urea cycle;
a protein involved in the electron transfer and/or oxidative phosphorylation (OxPhos) pathways;
a mitochondrial metabolism polypeptide; or
any combination thereof.

Embodiment 25. The kit of embodiment 23 or 24, wherein the one or more mitochondrial proteins is/are mitochondrial outer membrane protein(s).

Embodiment 26. The kit of any one of embodiments 23 to 25, wherein the one or more mitochondrial proteins is/are the one or more mitochondrial proteins listed in FIG. 3, or any combination thereof.

Embodiment 27. The kit of any one of embodiments 23 to 26, wherein the one or more mitochondrial protein is/are C1qBP and/or mitofusin 1 (Mfn1).

Embodiment 28. The kit of any one of embodiments 23 to 27, wherein said system autoimmune rheumatoid disease is selected from the group consisting of mixed connective tissue disease (MCTD), rheumatoid arthritis (RA), systemic lupus erythematosus (SLE), Sjögren's syndrome (SjS), systemic sclerosis (SSc), polymyositis (PM), and dermatomyositis (DM).

Embodiment 29. The kit of embodiment 23 to 28, wherein said system autoimmune rheumatoid disease is systemic lupus erythematosus (SLE).

Embodiment 30. The kit of embodiment 28 or 29, wherein the systemic lupus erythematosus (SLE) is accompanied by a secondary syndrome or disorder.

Embodiment 31. The kit of embodiment 30, wherein the secondary syndrome or disorder is antiphospholipid syndrome.

Embodiment 32. The kit of any one of embodiments 23 to 31, wherein the detecting of autoantibody levels is determined using an immunoassay.

Embodiment 33. The kit of any one of embodiments 23 to 32, wherein the subject is a human.

Embodiment 34. The kit of any one of embodiments 23 to 33, wherein the sample is blood, serum, or plasma.

Embodiment 35. The kit for use of any one of embodiments 23 to 34, further comprising reagents for detecting one or more autoantibodies known to be associated with the diagnosis or progression of said SARD.

Embodiment 36. The kit for use of any one of embodiments 23 to 35, wherein the kit is used in combination with the detection of one or more autoantibodies known to be associated with the identification, diagnosis or progression of said SARD.

Embodiment 37. The kit of embodiment 35 or 36, wherein the one or more autoantibodies known to be associated with the diagnosis or progression of said SARD is/are selected from the group consisting of anti-nuclear antibodies, anti-double stranded DNA antibodies, lupus anticoagulant antibodies, antiphospholipid antibodies, anticardiolipin antibodies, anti-D2 Glycoprotein I antibodies, anti-ribonucleoprotein antibodies, anti-small nuclear ribonucleoprotein antibodies, anti-Ro (SS/A) antibodies, anti-La (SS/B) antibodies, anti-mitochondrial DNA, anti-whole mitochondria, anti-mitochondrial RNA antibodies, and any combination thereof.

Embodiment 38. The kit for use of any one of embodiments 23 to 37, wherein the kit is used in combination with one or more criteria selected from one or more known diagnostic or classification measures of systemic autoimmune rheumatoid disease (SARD).

Embodiment 39. The kit of embodiment 38, wherein the one or more known diagnostic or classification measures is/are selected from the group consisting of American College of Rheumatology (ACR) classification criteria, Systemic Lupus International Collaborating Clinics (SLICC) classification criteria, Systemic Lupus Erythematosus Disease Activity Index (SLEDAI), lupus severity index (LSI), Systemic Lupus International Collaborating Clinics/American College of Rheumatology (SLICC/ACR) Damage Index (SDI), European League Against Rheumatism (EULAR) classification criteria, and criteria selected from FIGS. 10 and/or 11.

Embodiment 40. A method of producing a complex comprising one or more mitochondrial autoantigenic polypeptides and one or more autoantibodies specific to the respective one or more mitochondrial autoantigenic polypeptides; wherein said method comprises:
a. contacting a biological sample from a subject comprising one or more autoantibodies, with one or more mitochondrial autoantigenic polypeptides, wherein the subject has or is suspected to have a systemic autoimmune rheumatoid disease (SARD); and
b. detecting each complex between each respective autoantibody and mitochondrial autoantigenic polypeptide.

Embodiment 41. The method of embodiment 40, further comprising one or more features as defined in any one of embodiments 3 to 17.

Embodiment 42. A complex for use in the identification, diagnosis, or determination of progression of a systemic autoimmune rheumatoid disease (SARD) in a subject, wherein the complex comprises or is formed by one or more mitochondrial autoantigenic polypeptides and one or more autoantibodies specific to the respective one or more mitochondrial autoantigenic polypeptides.

Embodiment 43. A complex for use as a research tool in the detection of one or more autoantibodies specific to the one or more mitochondrial autoantigenic polypeptides in a biological sample, wherein the complex comprises one or more mitochondrial autoantigenic polypeptides and one or more autoantibodies specific to the respective one or more mitochondrial autoantigenic polypeptides.

Embodiment 44. A kit for use in the detection of autoantibodies in a biological sample, wherein the kit comprises autoantigens specific to the autoantibodies and reagents for the detection of the autoantibodies, wherein at least one of the autoantibodies are one or more autoantibodies specific to one or more mitochondrial autoantigenic polypeptides, and wherein at least one of the autoantigens are one or more mitochondrial autoantigenic polypeptides.

Embodiment 45. A kit for use in the detection of one or more autoantibodies specific to a respective one or more mitochondrial autoantigenic polypeptides in a biological sample, wherein the kit comprises one or more mitochondrial autoantigenic polypeptides specific to the respective one or more autoantibodies and reagents for the detection of the one or more autoantibodies.

Embodiment 46. A method of manufacturing the kit as defined in any one of embodiments 23 to 39, or 45, said method comprising:
a. immobilizing the one or more mitochondrial autoantigenic polypeptides; and
b. providing reagents for the detection of autoantibodies specific to the respective one or more mitochondrial autoantigenic polypeptides.

Embodiment 47. A method of manufacturing the kit as defined in embodiment 44, said method comprising:
a. immobilizing the autoantigens; and
b. providing reagents for the detection of autoantibodies specific to the respective autoantigens.

Embodiment 48. A method for producing or modifying a test for detecting systemic lupus erythematosus, the method comprising adding or integrating into said test quantifying a panel of mitochondrial autoantibodies, the panel comprising mitochondrial autoantibodies specific to one or more mitochondrial autoantigenic polypeptide.

Embodiment 49. The method of embodiment 48, wherein the one or more mitochondrial autoantigenic polypeptides comprise C1qBP and/or mitofusin 1 (Mfn1).

Embodiment 50. The method of embodiment 48 or 49, wherein the test is a blood, serum, or plasma test.

Other objects, advantages and features of the present description will become more apparent upon reading of the following non-restrictive description of specific embodiments thereof, given by way of example only with reference to the accompanying drawings.

### EXAMPLES

### Example 1: Materials and Methods

Human Serum Samples—Healthy Donors and Lupus Patients from the Systemic Autoimmune Rheumatic Disease Biobank and Database Repository (SARD-BDB)

This study was approved by the research ethics board of the CHUL (#B13-06-1243 and #B14-08-2108). Healthy volunteers, devoid of illnesses, infections or ongoing medications were recruited at the CHUde Québec—Université Laval (CHUL). Every patient met the ACR classification criteria for SLE [28]. Serum samples were obtained from peripheral blood punctures performed at the time of inclusion. In accordance with the Declaration of Helsinki, written consent was provided by each subject, and their clinical information and biological specimen were associated with an anonymized reference number. A convenience sample was used and no sample size calculation was done

### Clinical Data Collection

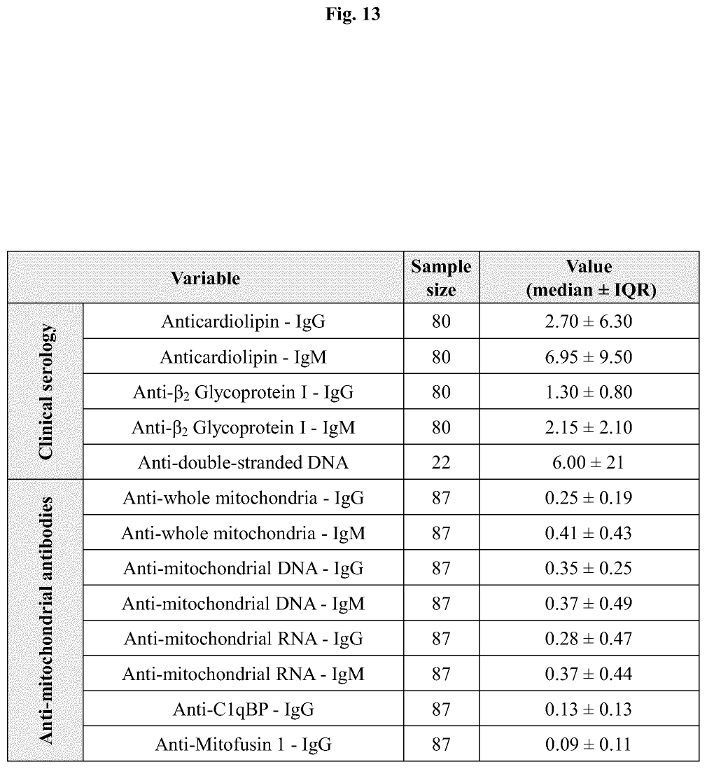

Information concerning sociodemographic (e.g. age, sex, ethnicity), clinical (e.g. classification criteria, clinical indexes, clinical manifestations, co-morbidities), laboratory (e.g. serology) and medication variables was gathered as previously reported [28]. These values are presented in FIGS. 11-13.

### Isolation and Preparation of Mitochondrial Samples

Intact mitochondria were isolated from C57BL/6J mouse livers, following previously published procedures [20]. Freshly isolated mitochondria were used for the panning of antibodies targeting the mitochondrial outer membrane (i.e., anti-whole mitochondria antibodies, AwMA). Dry-pelleted mitochondria were stored at −80° C. until needed. Frozen mitochondria were resuspended in hypotonic lysis buffer (10 mM HEPES, 2 mM KCl, 0.1% CHAPS, pH7.2) with protease inhibitors (cOmplete™ protease inhibitor cocktail; Roche, Basel, Switzerland) and underwent three cycles, alternating between thawing in a water bath (37° C.) and freezing in an ethanol-dry ice bath, followed by a 15 min sonication in an ice bath. Unbroken mitochondria were pelleted at 7,000×g for 10 min at 4° C. and discarded. Supernatant protein (i.e., mitochondrial lysates) content was determined by the bicinchoninic acid method (BCA protein assay kit. Thermo Fisher Scientific, Waltham, MA, USA). Nucleic acids were digested by addition of benzonase nuclease (Sigma-Aldrich, St-Louis, MO, USA. 100 U/mL, 30 min, 37° C.). Lysates were stored at −80° C. until use.

### Sample Preparation for Mass Spectrometry Identification of Mitochondrial Antigens Targeted by AMA in SLE

Optical densities at 405 nm (OD_{405 nm}) for 1:150 dilutions of serum samples from the SARD-BDB were previously assessed with our direct AwMA-ELISA [21] and a cut-off value for positivity to AwMA-IgG was calculated for OD_{405 nm} values>0.30. To account for inter-personal variabilities in autoantibody production, equal volumes of sera from either ten healthy donors (OD_{405 nm} values ranging from 0.08 to 0.17), or from ten SLE patients positive for AwMA (OD₄₀₅ nm values ranging from 0.68 to 3.00) were pooled and their IgG concentrations were determined by Human IgG total uncoated ELISA kit (Thermo Fisher Scientific). Experiments were performed in triplicate.

Immunoprecipitations: All incubations and 5-min washings used gentle rotary mixing, and were performed at ambient temperature (about 21° C.)—unless indicated. In all experiments, 3 mg of Dynabeads®-ProteinG were used. Before use, beads were washed three times with 1 mL PBS 1× (Wisent, Montréal, Qc, Canada). For retention of the beads in the tubes, a DynaMag™-2 (Thermo Fisher Scientific) magnetic strip was used.

Enrichment in mitochondrial outer membrane (MOM) antigens by panning: Intact mitochondria (0.5 mg as quantitated by BCA assay) were incubated overnight at 4° C. with diluted 1 mL sera (10%, in PBS with protease inhibitor cocktail) from pooled samples of healthy individuals or lupus patients. Non-bound serum components were removed by three 7000×g centrifugal washing, each performed for 10 min at 4° C., with 1.5 ml PBS. AwMA, bound with their outer membrane mitochondrial antigens were released by lysis in 1 mL lysis buffer with protease inhibitor, overnight at 4° C. to ensure the capture of IgGs by Dynabeads®-Protein G. Three washings were performed in PBS containing protease inhibitors, followed by two final washes with PBS devoid of protease inhibitors. Samples were resuspended in 100 μL ammonium bicarbonate buffer, pH 8.0 and stored at −80° C.

Negative controls: Dynabeads®-Protein G were incubated for 2 hrs an irrelevant monoclonal IgG (Clone IV.3, i.e. targeting FcγRIIa). Dynabeads®-bound IgG were then washed three times in 1.5 mL PBS and once in the same volume of lysis buffer. Samples were incubated overnight 4° C. with 1.5 ml of mitochondrial lysate brought to a protein concentration of 3 mg/mL. 1.5 mL washing were then performed as follows: thrice in protease inhibitors-containing lysis buffer, and twice in PBS. Beads were then resuspended in 100 μL of 75 mM ammonium bicarbonate pH 8.0 and stored at −80° C. until required.

### Identification of Mitochondrial Antigens Recognized by AMA

On-bead proteolysis: isolated samples were resuspended in 200 μL of 75 mM ammonium bicarbonate pH8.0 and supplemented with 2 g of a trypsin/Lys-C mix (Promega, Madison, WI, USA). Proteins in the samples were digested overnight at 37° C. on a rotating mixer. The digested products were acidified with trifluoroacetic acid and the peptides arising from the proteolytic digestion were isolated and washed on C18 tips (Thermo Fisher Scientific) according to the manufacturer's instructions. Finally, the purified peptides were dried by vacuum centrifugation and stored at −80° C.

LC-MS/MS analysis: Trypsin/Lys-C-digested peptides were separated on a Dionex Ultimate 3000 nano HPLC system coupled to a Q Exactive™ OrbiTrap™ mass spectrometer (Thermo Fisher). 10 μl of sample (a total of 1.5 μg) resuspended in 1% (vol/vol) formic acid were loaded with a constant flow of 4 l/min onto an Acclaim PepMap100 C18 column (0.3 mm id×5 mm; Dionex Corporation, Sunnyvale, CA, USA). After trap enrichment, peptides were eluted onto an EasySpray™ PepMap™ C18 nano column (75 μm×50 cm; Dionex Corporation) with a linear gradient of 8-40% solvent B (80% acetonitrile with 0.1% formic acid) over 240 min with a constant flow of 200 nl/min. The HPLC system was coupled to the mass spectrometer via an EasySpray™ source. The spray voltage was set to 2.0 kV and the temperature of the column set to 40° C. Full scan MS survey spectra (m/z 350-1600) in profile mode were acquired in the Orbitrap with a resolution of 70,000 after accumulation of 1,000,000 ions. The ten most intense peptide ions from the preview scan in the Orbitrap were fragmented by collision-induced dissociation (normalized collision energy 25% and resolution of 17,500) after the accumulation of 50,000 ions. Maximal filling times were 250 ms for the full scans and 60 ms for the MS/MS scans. Precursor ion charge state screening was enabled and all unassigned charge states as well as single, 7 and 8 charged species were rejected. The dynamic exclusion list was restricted to a maximum of 500 entries with a maximum retention period of 40 seconds and a relative mass window of 10 ppm. The lock mass option was enabled for survey scans to improve mass accuracy. Data were acquired using the XCalibur™ software (Thermo Fisher Scientific).

Mass spectrometry data analysis: Mass spectra data (.RAW files) were loaded into MaxQuant™ version 1.6.17.0 and searched against a protein database generated by merging the Homo sapiens and Mus musculus reference proteomes (Uniprot, versions 01-29-2021: 77,027 human and 55,470 mouse proteins) complemented with a list of common contaminants maintained by MaxQuant and concatenated with the reversed version of all sequences (decoy mode). The minimum peptide length was set to 7 amino acids and trypsin was specified as the protease allowing up to two missed cleavages. The mass tolerance was set to 7 ppm for the precursor ions and 20 ppm for the fragment ions. The following parameters were used: fixed carbamidomethylation of cysteine (+57.0214 Da), oxidation of methionine (+15.9949 Da) as a variable modification and a peptide-spectrum and protein match false-discovery rate of 0.01.

Intensity-based label-free quantification (LFQ) values were used to estimate the relative abundance of proteins in each replicate groups, which correspond to the sum of all peak intensities over all tandem mass spectra assigned to a particular protein. Missing LFQ data imputation was estimated by using a noise value corresponding to the lowest 1% percentile of the LFQ distribution. This noise value was imputated for each sample when the intensity value is missing (i.e., undetected proteins) and selected as the minimum LFQ intensity for low abundance protein identifications for which LFQ values fall below the 1% percentile background.

Filtering and data analysis of proteomic data: Matches to reverse proteins, proteins with zero intensity values in all datasets, proteins with negative MaxQuant scores and common contaminants were removed from the final protein repertoire. Identified proteins were cross-referenced using their Uniprot ID for their cellular localization and, when the information was available, for their mitochondrial sublocalization. Pathways involving the mitochondrial proteins identified were analyzed, using the Reactome pathway knowledge base and the Database for Annotation, Visualization and Integrated Discovery (DAVID).

Identification of Mitochondrial Antigens Recognized by AMA

Detection of IgG targeting several mtAgs were performed as previously described [20-22] with the following modification: microplates were coated overnight at 4° C. with 225 ng protein in 25 μL PBS 1× (i.e. 9 ng/μL). The following proteins were tested: C1qBP (Novus Biologicals, Centennial, CO, USA), Mfn1 (Origene Technologies, Rockville, MD, USA), ATP5F1B (Cusabio, Wuhan, China) and OTC. Other mitochondrial proteins were also tested (healthy: n=10; SLE n=30): CPS 1, NAGS, ETFA (Origene Technologies), GOT2 (BioVision Inc., Milpitas, California, USA), ALDH2 (RayBiotech Life, Peachtree Corners, GA, USA). OTC, CPS 1 and NAGS were provided by Dr. Rubio [29,30].

### Statistical Analyses

Sociodemographic, clinical and laboratory values are presented as either median±inter-quartile range (IQR), or n (%), or as mean±SD. Comparisons between groups were performed using the Wilcoxon-Mann-Whitney test. Spearman correlations were calculated to see associations between AMA and antibodies assessed in clinical serologies. Youden's index was determined to set a cut-off value for positivity to anti-C1qBP and anti-Mfn1. Logistic regressions, adjusted for sex and age were performed and expressed as their adjusted odds ratio (aOR), 95% confidence interval (95% CI) and p-value. Statistical analyses were performed with Prism 9 software (GraphPad Software Inc., La Jolla, CA, USA), SAS version 9.4 (SAS Institute Inc., Cary, NC, USA), and figures were assembled with Photoshop CC 2019 version 20.0.4 (Adobe Systems Inc.).

### Example 2: Results

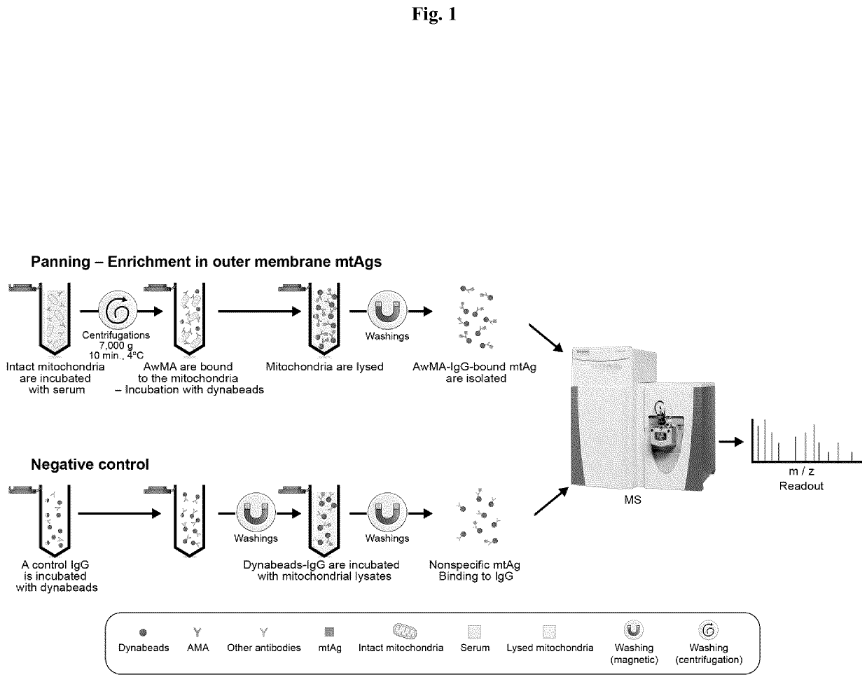

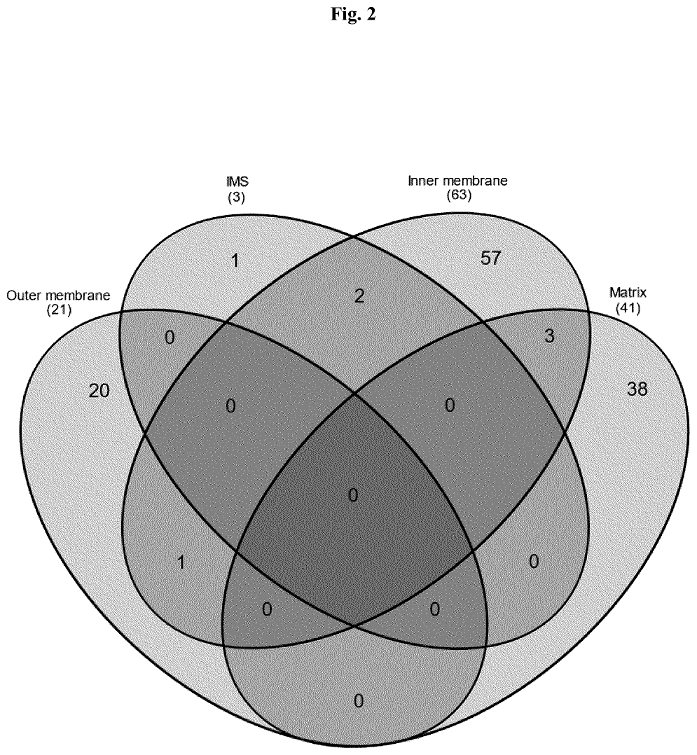

We used whole (i.e., intact) mitochondria to capture AMA recognizing components from the mitochondrial outer membrane (MOM). An irrelevant antibody, targeting a protein absent in mitochondria, was also utilized as control, to appreciate both the non-specific binding and potential endogenous proteins interacting with IgG (e.g., the complement pathway). These approaches are illustrated in FIG. 1. We assessed the localizations of the proteins identified to identify members of the mitochondrial proteome. 173 proteins were assigned to the mitochondrion, 51 of which lacked references pertaining to their mitochondrial sublocalization. 41 proteins were expressed within the MM, 3 in the IMS, 63 in the MIM and 21 in the MOM (FIG. 2). Of note, 6 proteins were associated with more than one mitochondrial compartment (FIGS. 2, 3 and 4). When grouped for protein interactions, we observed an enrichment of three networks in SLE samples: the C1q complement system, the serin protease inhibitors (serpin) superfamily, and members of the pyruvate dehydrogenase complex (PDC) (FIGS. 4 and 5). Analysis through gene ontology indicated the mitochondrion as the most significant subcellular structures associated to the proteins identified, and the mitochondrial metabolism as the functions/processes with the highest significances (FIG. 5).

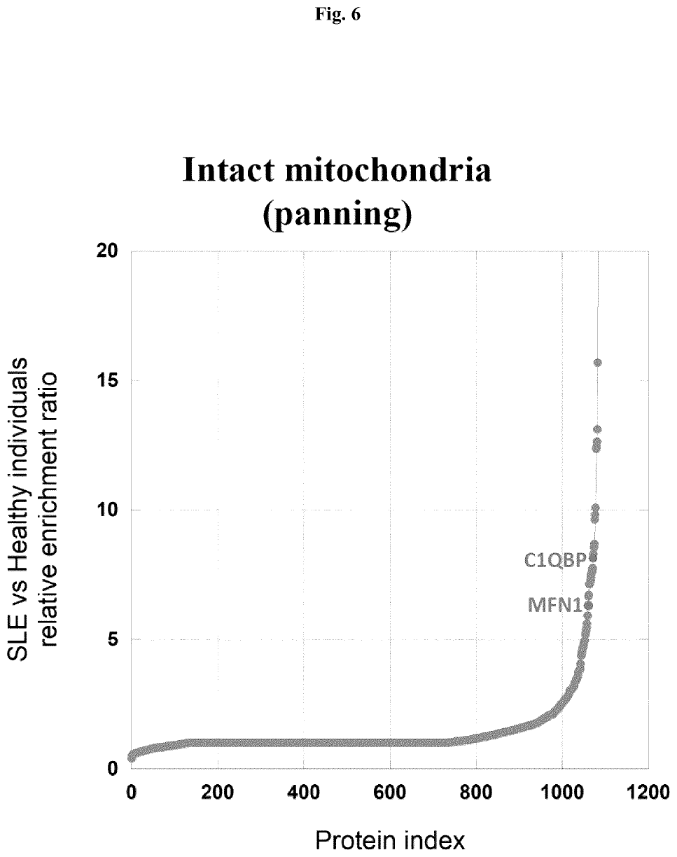

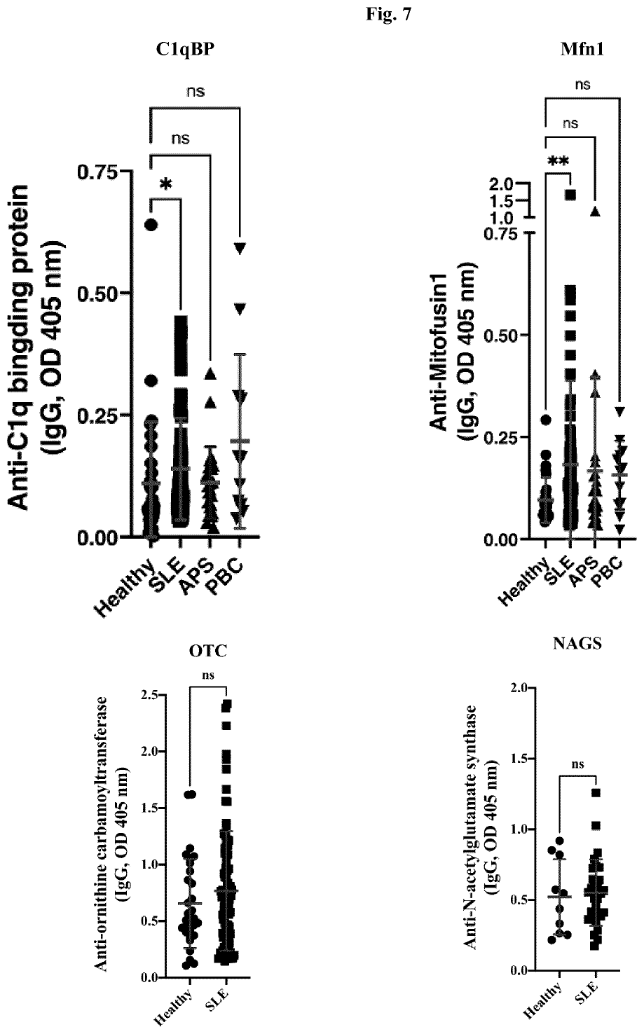

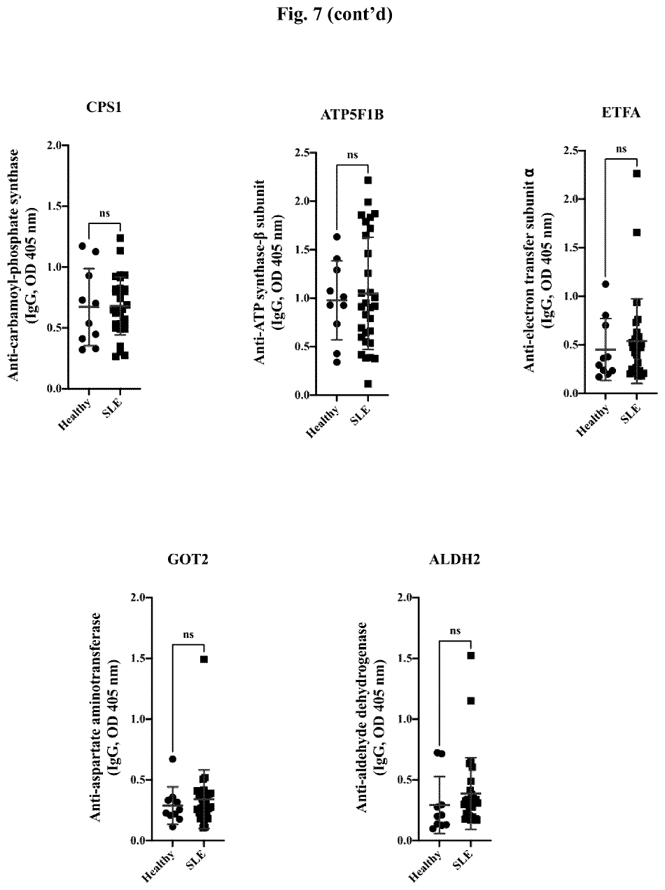

The complement component 1 Q subcomponent-binding protein (C1qBP) is a mitochondrial protein belonging to the C1q network that is stored within the mitochondrion and can be then addressed to the surface of the plasma membrane and/or shed into the extracellular space [31]. Thus, although C1qBP is not strictly a mitochondrial protein, its enrichment in SLE samples, identification in our proteomic analyses and its relevance to mitochondria and complement stimulated further analyses (FIGS. 2-6). By testing the immunoreactivity of sera from SLE patients to C1qBP by direct ELISA, we observed that IgGs against C1qBP were significantly elevated in SLE patients, in comparison with healthy individuals (p=0.049. FIG. 7).

While antibodies in primary biliary cirrhosis (PBC) target proteins from the mitochondria inner membrane (MIM), inter-membrane space (IMS) or the matrix (MM) [19,32-34], previous findings determined that antibodies could recognize components on the surface of the outer membrane in SLE [20,22,35-37]. A protein from the MOM, associated to the PDC but distinct from the antigenic targets of AMA-M2 and presenting an elevated score in our panning approach was selected—mitofusin-1 (Mfn1) (FIGS. 2-6). For comparisons, we tested other mitochondrial proteins available, with MIM or MM locations that were identified by our proteomic approaches in both healthy and SLE samples from a subset of patients having highly diverse sociodemographic and clinical characteristics (FIGS. 11 and 12). Three mitochondrial representatives of the urea pathways [i.e. ornithine carbamoyltransferase (OTC), carbamoyl-phosphate synthase (CPS1) and N-acetylglutamate synthase (NAGS)], two proteins implicated in electron transfer and oxidative phosphorylation [i.e. the beta subunit of the MIM complex ATP synthase (ATP5F1B) and electron transfer flavoprotein subunit alpha (ETFA)] as well as two proteins involved in metabolic processes [i.e. aspartate aminotransferase (GOT2) and aldehyde dehydrogenase (ALDH2)] were selected. When used as coating antigens in direct ELISAs, IgG against Mfn-1 (p=0.0044) were significantly increased, in comparison with healthy individuals. Antibodies against the aforementioned control proteins were detected and a trending elevation in SLE compared with healthy donors was observed (FIG. 7).

Interestingly, anti-Mfn-1 and anti-C1qBP IgG antibody levels were not significantly elevated in other autoimmune diseases compared to healthy controls, such as antiphospholipid syndrome (APS) and PBC (FIG. 7). Specifically, in PBC, anti-mitochondrial antibodies, particularly anti-mitochondrial proteins (e.g., sulfite oxidase, glycogen phosphorylase, PDC-E2) have been previously shown to be present in these patients (28). These data suggest that certain anti-mitochondrial protein antibodies, like anti-Mfn-1 and anti-C1qBP antibodies, may be specifically and strongly associated with SLE.

Next, we determined if the autoantibodies to either of these two components, C1qBP and Mfn-1, were associated with a definable cytoplasmic immunofluorescent staining pattern on routine HEp-2 substrates, as seen with antibodies to pyruvate dehydrogenase complex in PBC serum (28, 51). Indirect immunofluorescence labelling of HEp-2 cells with a commercial antibody against C1qBP produced intense speckled staining of the nuclear region and a lower signal in the cytosol. Conversely, commercial anti-Mfn-1 displayed reticular staining of the cytoplasm, typical of AMAs (FIG. 8A). AwMA-positive SLE patient serum displayed a wide variety of patterns, but none qualified as positive for mitochondrial staining. In contrast, serum from PBC patients analyzed using the same approach revealed an obvious cytosolic pattern, with no nuclear staining when observed using a regular fluorescence microscope. However, when the same slides were examined using a confocal microscope, the autoantibodies from 7 out of the 9 SLE patients tested displayed various intensities of cytoplasmic staining, reminiscent of those observed in samples from patients with PBC or observed using commercial antibodies against C1qBP and Mfn-1 (FIG. 8B).

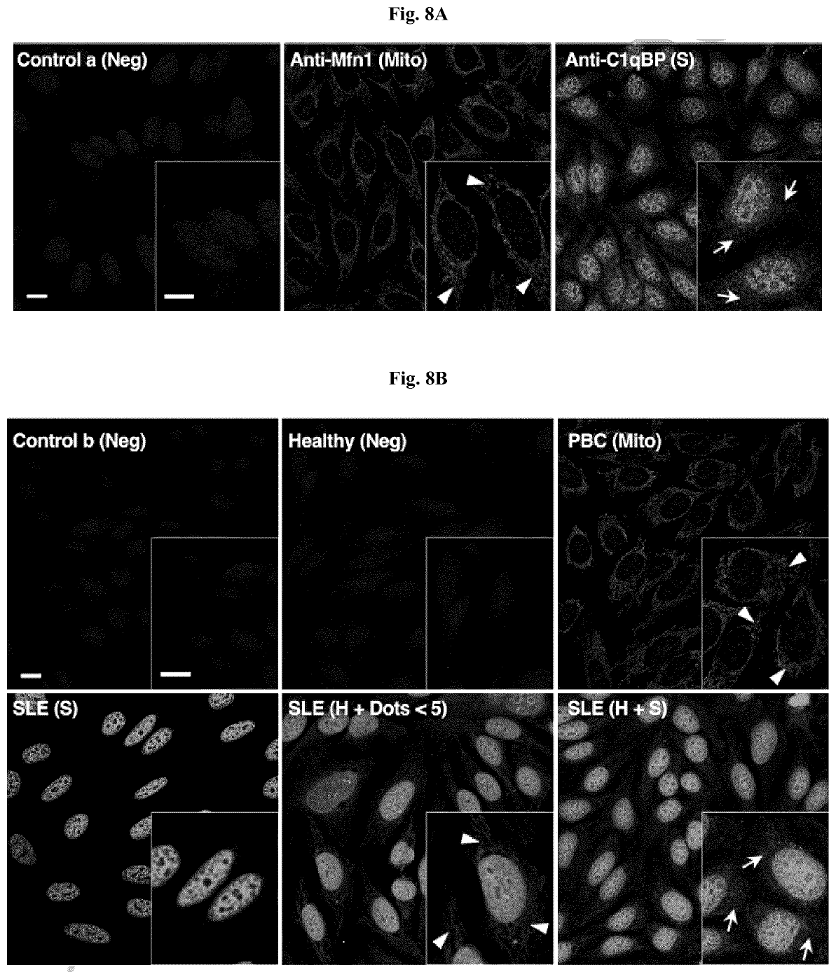

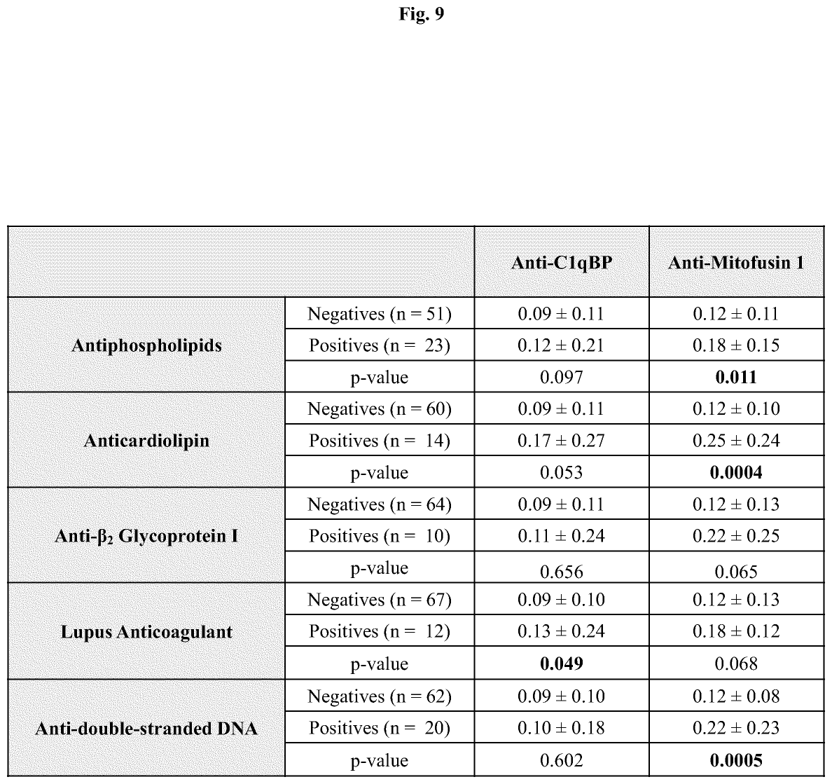

We then investigated the biostatistical associations of C1qBP and Mfn-1 with various disease parameters. When associated to SLE autoantibodies routinely assessed by clinical laboratories (FIG. 9), anti-C1qBP IgGs were significantly increased in patients positive for the lupus anticoagulant (LA; p=0.049), while anti-Mfn1 IgGs were increased in patients with positivity to antiphospholipids (p=0.011), as well as anti-double-stranded DNA autoantibodies (i.e., anti-dsDNA; p=0.0005). When considering individual IgGs, anti-Mfn1 were increased in patients positive for anticardiolipin (p=0.0004) and neared significance in patients with anti-β₂ glycoprotein I (i.e., anti-β₂GPI; p=0.065). These results were further confirmed by the correlations between OD_{405 nm} measured for anti-Mfn1 and continuous variables available (i.e., levels) for anticardiolipin (rₛ=0.454; p<0.0001), anti-β₂GPI (rₛ=0.261; p=0.019) and anti-dsDNA (rₛ=0.443; p=0.039) (FIG. 10). Interestingly, anti-dsDNA antibodies are highly specific to SLE and have been shown to be associated with development of lupus nephritis (36. 89). Anti-cardiolipin antibodies, however, have been shown to be strongly associated with venous/arterial thrombosis in SLE (62,72).

When matched with levels of other AMA, levels of anti-C1qBP were associated with both AwMA-IgG (rₛ=0.319; p=0.003) and AmtDNA-IgG (rₛ=0.234; p=0.029). Moreover, while anti-Mfn1 also displayed strong correlations with AwMA-IgG (rₛ=0.657; p<0.0001) and AmtDNA-IgG (rₛ=0.518; p<0.0001), they also correlated with all the AMA assessed in the SARD-BDB. Of note, levels of anti-C1qBP also correlated with those of anti-Mfn1 (rₛ=0.49; p>0.0001).

FIG. 12 characterizes the clinical manifestations of each SLE patient from this study. Of note, a high incidence of several clinical manifestations, such as anti-double-stranded DNA [57%], renal disorders [27.9%], and haematological disorders [80.%]) was observed in these SLE patients, which were shown to have elevated anti-Mfn-1 and anti-C1qBP antibody levels (FIG. 7). Conversely, a lower incidence rate of certain manifestations, such as neurological disorders or manifestations (4.7%), was observed in these patients.

Taken together, these data suggest the potential of anti-Mfn-1 and anti-C1qBP autoantibodies as potent and specific biomarkers for diagnosing SLE, as well as for determining SLE disease severity and progression.

### Example 3: Discussion

Patients with SLE display antibodies from various subclasses (e.g., IgG, IgM, IgA) against a wide array of self-antigens [38]. The epitopes targeted by these autoantibodies comprise, but are not limited to, phospholipids [e.g., aPL, aCL, LA] [39], phospholipid-protein complexes [e.g., anti-β₂ glycoprotein I (anti-β₂ GPI)] [40], nucleic acids (e.g., dsDNA, AmtDNA, AmtRNA) [20,21] and associated proteins [e.g., anti-nuclear antibodies (ANA), transcription factors and ribonucleoproteins] [41] among others [42]. During the course of the disease, cell death and/or activation was reported to trigger the release of mitochondrial structures (e.g., mtDNA, cardiolipin) or even whole functional organelles [7,9,10,12]. The pro-inflammatory influence of mitochondrial DAMPs on the innate immune system is extensively documented in homeostasis and SLE pathophysiology [18]. Furthermore, distinct autoantibodies targeting various types of mitochondrial biomolecules such as phospholipids (i.e., aCL targeting cardiolipin) [43], nucleic acids (i.e., mtDNA, mtRNA) [20,21] and antigens whose precise nature remains to be characterized (i.e., AMA-M5) were reported in SLE [37].

While various proteins involved in the mitochondrial processing of pyruvate (e.g., PDC-E2), sulfite oxidase and glycogen phosphorylase are mitochondrial proteins known to be targeted by anti-mitochondrial antibodies (i.e., respectively by AMA-M2, -M4 and -M9) in primary biliary cirrhosis [19], limited knowledge is available concerning the extent of the mitochondrial proteome targeted by AMA in SLE [2]. To date, the only mitochondrial protein with autoantibodies associated with disease manifestations in SLE is HSP60 [44]. Herein, we used several approaches to enrich mitochondrial antigens in sample in our mass spectrometry analyses. In the experimental design, we also included a control samples that included an irrelevant antibody intended to identify non-specific binding. These approaches indicated the presence of the complement pathways (i.e., known to be targeted by circulating autoantibodies in SLE while also interacting with IgG and IgM [45]), suggesting that, one should consider these entities with caution as—additionally to their potential antigenicities—they can be co-isolated with autoantibodies. It is noteworthy that our analyses highlighted the presence of non-mitochondrial proteins (e.g., ficolin-3, serpins) that may be of interest in SLE. However, we restricted the focus of or study on the proteins assigned to the mitochondrial proteome. While we tested a handful of mitochondrial proteins, systematic characterization of patients' immunoreactivities to large subsets of mitochondrial antigens would be enhanced by the use of high-throughput screening methods such as nucleic acid programmable protein array (NAPPA). In addition, the present study focuses on IgG while other antibody classes such as IgM or IgA may also have an impact on SLE pathophysiology. These autoantibodies may be studied by using similar approaches with optimized isolation steps, depending on the antibody class assessed.

Two proteins with significant immunogenicities stood out from the various candidates assessed due to their increase in SLE samples. First, C1qBP, a protein stored within the mitochondrion and dispatched to the MOM and/or the cell membrane, or secreted into the circulation [48,49], where it may be targeted by circulating AMA. Second, Mfn1, a protein embedded in the MOM that could be recognized by AMA upon the release of whole mitochondria into the extracellular space. We speculate that C1qBP potentially constitutes a circulating autoantigen enabling the formation of protein-protein scaffolds with C1q and/or IgGs allowing pro-inflammatory signaling (e.g., respectively through the classical complement pathway and signaling by Fcγ receptors). Levels of anti-C1qBP were associated with those of IgGs targeting whole mitochondria, mtDNA and Mfn1, while anti-mitofusin 1 were correlated with all AMA assessed. In addition, the strong correlation between anti-Mfn1 and AwMA suggests that mitofusin 1 might represent one of the main mtAg of the MOM. Moreover, our previous work on AMA in SLE and the correlations of AMA with circulating mtDNA or anticardiolipin showcase the mitochondrion as a reservoir of various immunogenic biomolecules [14,20-22].

Patients with SLE display antibodies from various subclasses (e.g. IgG, IgM, and IgA) against a wide array of self-antigens [60]. The epitopes targeted by these autoantibodies comprise, but are not limited to, phospholipids (e.g. aPL, aCL, lupus anticoagulant [LA]) [61-63], phospholipid-protein complexes (e.g. anti-β₂ glycoprotein I [anti-β₂GPI]) [64], nucleic acids (e.g. anti-double-stranded DNA [dsDNA], AmtDNA, and AmtRNA) [27,28] and associated proteins (e.g. anti-nuclear antibodies [ANA], transcription factors, and ribonucleoproteins) [65] among others [66]. During the course of the disease, cell death and/or activation was reported to trigger the release of mitochondrial structures (e.g. mtDNA, cardiolipin) or even whole functional organelles [9-18]. The pro-inflammatory influence of mitochondrial DAMPs on the innate immune system is extensively documented in homeostasis and SLE pathophysiology [25,67-71]. Furthermore, distinct autoantibodies targeting various types of mitochondrial biomolecules such as phospholipids (i.e. aCL targeting cardiolipin) [72,73], nucleic acids (i.e. mtDNA, mtRNA) [27,28], and antigens whose precise nature remains to be characterized (i.e. AMA-M5) were reported in SLE [54,55].

Due to the nature of the patient recruitment, the samples available were collected at the time of patient inclusion into the cohort, which does not necessarily reflect the pattern of antibodies prevalent at the time of diagnosis. Moreover, the ethnic distribution of the patients and the relatively low clinical scores of the patients at the time of the phlebotomy may have influenced the various data assessed. Thus, future studies using samples from large inception cohorts should be dedicated to assessing the associations between anti-C1qBP-IgG and anti-Mfn1-IgG with disease manifestations in order to appreciate their qualities as biomarkers in SLE.

In conclusion, anti-mitochondrial autoantibodies and their specific antigens constitute potential candidates for the development of novel assays allowing improvements to diagnosis, prognosis and/or patient stratification in SLE with IgGs against C1qBP and Mfn1 as promising candidates.

### REFERENCES

1. Rambold A S, Pearce E L. Mitochondrial dynamics at the interface of immune cell metabolism and function. Trends Immunol 39: 6-18, 2018.
2. Veltri K L, Espiritu M, Singh G. Distinct genomic copy number in mitochondria of different mammalian organs. J Cell Physiol 143: 160-164, 1990.
3. Liesa M, Shirihai O S. Mitochondrial dynamics in the regulation of nutrient utilization and energy expenditure. Cell Metab 17: 491-506, 2013.
4. Palmfeldt J, Bross P. Proteomics of human mitochondria. Mitochondrion 33: 2-14, 2017.
5. Rorbach J, Minczuk M. The post-transcriptional life of mammalian mitochondrial RNA. Biochemical J 444: 357-373, 2012.
6. Mercer T R, Neph S, Dinger M E, et al. The human mitochondrial transcriptome. Cell 146: 645-58, 2011.
7. Calfee C S, Matthay M A. Clinical immunology: Culprits with evolutionary ties. Nature. 464: 41-42, 2010.
8. Andersson S G, Zomorodipour A, Andersson J O, et al. The genome sequence of Rickettsia prowazekii and the origin of mitochondria. Nature 396: 133-140, 1998.
9. McDonald B, Pittman K, Menezes G B, et al. Intravascular danger signals guide neutrophils to sites of sterile inflammation. Scienc 330: 362-366, 2010.
10. Garcia-Martinez I, Santoro N, Chen Y, et al. Hepatocyte mitochondrial DNA drives nonalcoholic steatohepatitis by activation of TLR9. J Clin Invest 126: 859-64, 2016.
11. Marques P E, Amaral S S, Pires D A, et al. Chemokines and mitochondrial products activate neutrophils to amplify organ injury during mouse acute liver failure. Hepatology 56: 1971-1982, 2012.
12. Oka T, Hikoso S, Yamaguchi 0, et al. Mitochondrial DNA that escapes from autophagy causes inflammation and heart failure. Nature 485: 251-255, 2012.
13. Yousefi S, Mihalache C, Kozlowski E, Schmid I, Simon H U. Viable neutrophils release mitochondrial DNA to form neutrophil extracellular traps. Cell Death Differ 16: 1438-1444. 2009.
14. Lood C, Blanco L P, Purmalek M M, et al. Neutrophil extracellular traps enriched in oxidized mitochondrial DNA are interferogenic and contribute to lupus-like disease. Nat Med 22: 146-153, 2016;
15. Caielli S, Athale S, Domic B, et al. Oxidized mitochondrial nucleoids released by neutrophils drive type I interferon production in human lupus. J Exp Med 213: 697-713, 2016.
16. Yousefi S, Gold J A, Andina N, et al. Catapult-like release of mitochondrial DNA by eosinophils contributes to antibacterial defense. Nat Med 14: 949-253, 2008.
17. Boudreau L H, Duchez A C, Cloutier N, et al. Platelets release mitochondria serving as substrate for bactericidal group IIA-secreted phospholipase A2 to promote inflammation. Blood 124: 2173-2183, 2014.
18. Zhang B, Asadi S, Weng Z, Sismanopoulos N, Theoharides T C. Stimulated human mast cells secrete mitochondrial components that have autocrine and paracrine inflammatory actions. PLoS One 7: e49767, 2012.
19. Maeda A, Fadeel B. Mitochondria released by cells undergoing TNF-alpha-induced necroptosis act as danger signals. Cell Death Dis 5: e1312, 2014.
20. Melki I, Allaeys I, Tessandier N, et al. Platelets release mitochondrial antigens in systemic lupus erythematosus. Sci Transl Med 13: eaav5928, 2021.
21. Sun C, Liu X, Wang B, et al. Endocytosis-mediated mitochondrial transplantation: Transferring normal human astrocytic mitochondria into glioma cells rescues aerobic respiration and enhances radiosensitivity. Theranostics 9: 3595-3607, 2019.
22. Sinha P, Islam M N, Bhattacharya S, Bhattacharya J. Intercellular mitochondrial transfer: Bioenergetic crosstalk between cells. Curr Opin Genet Dev 38: 97-101, 2016.
23. Nicolás-Ávila J A, Lechuga-Vieco A V., Esteban-Martinez L, et al. A network of macrophages supports mitochondrial homeostasis in the Heart. Cell 183: 94-109, 2020.
24. Davis CHO, Kim K Y, Bushong E A, et al. Transcellular degradation of axonal mitochondria. Proc Natl Acad Sci USA 111: 9633-9638, 2014.
25. Krysko D V, Agostinis P, Krysko 0, et al. Emerging role of damage-associated molecular patterns derived from mitochondria in inflammation. Trends Immunol 32: 157-164, 2011.
26. Berg P A, Klein R. Mitochondrial antigens and autoantibodies: from anti-M1 to anti-M9. Klin Wochenschr 64: 897-909, 1986.
27. Becker Y, Loignon R C, Julien A S, et al. Anti-mitochondrial autoantibodies in systemic lupus erythematosus and their association with disease manifestations. Sci Rep 9: 4530, 2019.
28. Becker Y, Marcoux G, Allaeys I, et al. Autoantibodies in systemic lupus erythematosus target mitochondrial RNA. Front Immunol 10: 1026, 2019.
29. Becker Y L C, Julien A-S, Godbout A, Boilard F, Fortin P R. Pilot study of anti-mitochondrial antibodies in antiphospholipid syndrome. Lupus 29: 1623-1629, 2020.
30. Bell C, English L, Boulais J, et al. Quantitative proteomics reveals the induction of mitophagy in tumor necrosis factor-α-activated (TNFα) macrophages. Mol Cell Proteomics 12: 2394-2407, 2013.
31. Bueno M, Zank D, Buendia-Roldin I, et al. PINK1 attenuates mtDNA release in alveolar epithelial cells and TLR9 mediated profibrotic responses. PLoS One 14: e0218003, 2019.
32. Matheoud D, Sugiura A, Bellemare-Pelletier A, et al. Parkinson's Disease-related proteins PINK1 and parkin repress mitochondrial antigen presentation. Cell. 166: 314-327, 2016.
33. Dabhi V M, Lindahl K F. Short peptides sensitize target cells to CTL specific for the MHC class Ib molecule, H2-M3. Eur J Immunol 28: 3773-3782, 1998.
34. Qi L, Rojas J-M, Ostrand-Rosenberg S. Tumor cells present MHC class II-restricted nuclear and mitochondrial antigens and are thepredominant antigen presenting cells in vivo. J Immunol 165: 5451-5461, 2000.
35. Reimer G, Rubin R L, Kotzin B L, Tan E M. Anti-native DNA antibodies from autoimmune sera also bind to DNA in mitochondria. J Immunol 133: 2532-2536, 1984.
36. de Leeuw K, Bungener L, Roozendaal C, Bootsma H, Stegeman C A. Auto-antibodies to double-stranded DNA as biomarker in systemic lupus erythematosus: Comparison of different assays during quiescent and active disease. Rheumatology 56: 698-703, 2017.
37. Craig M L, Bankovich A J, McElhenny J L, Taylor R P. Clearance of anti-double-stranded DNA antibodies: the natural immune complex clearance mechanism. Arthritis Rheum 43: 2265-2275, 2000.
38. Marcoux G, Duchez A C, Rousseau M, et al. Microparticle and mitochondrial release during extended storage of different types of platelet concentrates. Platelets 28: 272-280, 2017.
39. Marcoux G, Magron A, Sut C, et al. Platelet-derived extracellular vesicles convey mitochondrial DAMPs in platelet concentrates and their levels are associated with adverse reactions. Transfusion 59: 2403-2414, 2019.
40. Dieudé M, Senécal J L, Raymond Y, Dieude M, Senecal J L, Raymond Y. Induction of endothelial cell apoptosis by heat-shock protein 60-reactive antibodies from anti-endothelial cell autoantibody-positive systemic lupus erythematosus patients. Arthritis Rheum 50: 3221-3231, 2004.
41. Dieudé M, Correa J A, Neville C, et al. Association of autoantibodies to heat-shock protein 60 with arterial vascular events in patients with antiphospholipid antibodies. Arthritis Rheum 63: 2416-2424, 2011.
42. Tan E M, Cohen A S, Fries J F, et al. The 1982 revised criteria for the classification of systemic lupus erythematosus. Arthritis Rheum 25: 1271-1277, 1982.
43. Hochberg M C. Updating the American College of Rheumatology revised criteria for the classification of systemic lupus erythematosus. Arthritis Rheum 40: 1725, 1997.
44. Piette J C. Updating the American College of Rheumatology criteria for systemic lupus erythematosus: comment on the letter by Hochberg. Arthritis Rheum 41: 751, 1998.
45. Heberle H, Meirelles V G, da Silva F R, Telles G P, Minghim R. InteractiVenn: A web-based tool for the analysis of sets through Venn diagrams. BMC Bioinformatics 16: 169, 2015.
46. Fabregat A, Sidiropoulos K, Viteri G, et al. Reactome pathway analysis: a high-performance in-memory approach. BMC Bioinformatics 18: 142, 2017.
47. Jassal B, Matthews L, Viteri G, et al. The reactome pathway knowledgebase. Nucleic Acids Res 48(D1): D498-D503, 2020.
48. Huang D W, Sherman B T, Lempicki R A. Systematic and integrative analysis of large gene lists using DAVID bioinformatics resources. Nat Protoc 4: 44-57, 2009.
49. Huang D W, Sherman B T, Lempicki R A. Bioinformatics enrichment tools: Paths toward the comprehensive functional analysis of large gene lists. Nucleic Acids Res 37: 1-13, 2009.
50. Tomizawa M, Shinozaki F, Fugo K, et al. Anti-mitochondrial M2 antibody-positive autoimmune hepatitis. Exp Ther Med 10: 1419-1422, 2015.
51. Klein R, Berg P A. Anti-M4 antibodies in primary biliary cirrhosis react with sulphite oxidase, an enzyme of the mitochondrial inter-membrane space. Clin Exp Immunol 84: 445-448, 1991.
52. Berg P A, Klein R. Mitochondrial antigen/antibody systems in primary biliary cirrhosis: revisited. Liver 15: 281-292, 1995.
53. Labro M T, Andrieu M C, Weber M, Homberg J C. A new pattern of non-organ- and non-species-specific anti-organelle antibody detected by immunofluorescence: the mitochondrial antibody number 5. Clin Exp Immunol 31: 357-366, 1978.
54. Meroni P L, Harris E N, Brucato A, et al. Anti-mitochondrial type M5 and anti-cardiolipin antibodies in autoimmune disorders: studies on their association and cross-reactivity. Clin Exp Immunol 67: 484-491, 1987.
55. La Rosa L, Covini G, Galperin C, et al. Anti-mitochondrial M5 type antibody represents one of the serological markers for anti-phospholipid syndrome distinct from anti-cardiolipin and anti-b2-glycoprotein I antibodies. Clin Exp Immunol 112: 144-151, 1998.
56. Hosszu K K, Valentino A, Peerschke E I, Ghebrehiwet B. SLE: novel postulates for therapeutic options. Front Immunol 11: 583853, 2020.
57. Ghebrehiwet B, Hosszu K K, Valentino A, Ji Y, Peerschke E I B. Monocyte expressed macromolecular C1 and C1q receptors as molecular sensors of danger: Implications in SLE. Front Immunol 5: 278, 2014.
58. Hosszu K K, Valentino A, Vinayagasundaram U, et al. D C-SIGN, C1q, and gC1qR form a trimolecular receptor complex on the surface of monocyte-derived immature dendritic cells. Blood 120: 1228-1236, 2012.
59. Ghebrehiwet B, Geisbrecht B V., Xu X, Savitt A G, Peerschke E I B. The C1q receptors: Focus on gC1qR/p33 (C1qBP, p32, HABP-1)1. Semin Immunol 45: 101338, 2019.
60. Dema B, Charles N. Autoantibodies in SLE: specificities, isotypes and receptors. Antibodies 5: 2, 2016.
61. Hudson M, Herr A L, Rauch J, et al. The presence of multiple prothrombotic risk factors is associated with a higher risk of thrombosis in individuals with anticardiolipin antibodies. J Rheumatol 30: 2385-2391, 2003.
62. Neville C, Rauch J, Kassis J, et al. Thromboembolic risk in patients with high titre anticardiolipin and multiple antiphospholipid antibodies. Thromb Haemost 90: 108-115, 2003.
63. Levine J S, Branch D W, Rauch J. The antiphospholipid syndrome. N Engl J Med 346: 752-763, 2002.
64. Subang R, Levine J S, Janoff A S, et al. Phospholipid-bound β2-glycoprotein I induces the production of anti-phospholipid antibodies. J Autoimmun 15: 21-32, 2000.
65. Pisetsky D S, Lipsky P E. New insights into the role of antinuclear antibodies in systemic lupus erythematosus. Nat Rev Rheumatol 16: 565-579, 2020.
66. Yaniv G, Twig G, Shor D B A, et al. A volcanic explosion of autoantibodies in systemic lupus erythematosus: a diversity of 180 different antibodies found in SLE patients. Autoimmun Rev 14: 75-79, 2015.
67. Wenceslau C F, McCarthy C G, Szasz T, et al. Mitochondrial damage-associated molecular patterns and vascular function. Eur Heart J 35: 1172-1177, 2014.
68. He J, Lu Y, Xia H, et al. Circulating mitochondrial DAMPs are not effective inducers of proteinuria and kidney injury in rodents. PLoS One 10: e0124469, 2015.
69. Yasui K, Matsuyama N, Kuroishi A, Tani Y, Furuta R A, Hirayama F. Mitochondrial damage-associated molecular patterns as potential proinflammatory mediators in post-platelet transfusion adverse effects. Transfusion 56: 1201-1212, 2016.
70. Simmons J D, Lee Y L L, Pastukh V M, et al. Potential contribution of mitochondrial DNA damage associated molecular patterns in transfusion products to the development of acute respiratory distress syndrome after multiple transfusions. J Trauma Acute Care Surg 82: 1023-1029, 2017.
71. Chakraborty K, Raundhal M, Chen B B, et al. The mito-DAMP cardiolipin blocks IL-10 production causing persistent inflammation during bacterial pneumonia. Nat Commun 8: 13944, 2017.
72. Wahl D G, Guillemin F, de Maistre E, Perret C, Lecompte T, Thibaut G. Risk for venous thrombosis related to antiphospholipid antibodies in systemic lupus erythematosus—a meta-analysis. Lupus 6: 467-473, 1997.
73. Love P E, Santoro S A. Antiphospholipid antibodies: Anticardiolipin and the lupus anticoagulant in systemic lupus erythematosus (SLE) and in non-SLE disorders. Prevalence and clinical significance. Ann Intern Med 112: 682-698, 1990.
74. Klein R, Maisch B, Kochsiek K, Berg P A. Demonstration of organ specific antibodies against heart mitochondria (anti-M7) in sera from patients with some forms of heart diseases. Clin Exp Immunol 58: 283-292, 1984.
75. Cicek G, Schiltz E, Hess D, Staiger J, Brandsch R. Analysis of mitochondrial antigens reveals inner membrane succinate dehydrogenase flavoprotein subunit as autoantigen to antibodies in anti-M7 sera. Clin Exp Immunol 128: 83-87, 2002.
76. Otto A, Stahle I, Klein R, Berg P A, Pankuweit S, Brandsch R. Anti-mitochondrial antibodies in patients with dilated cardiomyopathy (anti-M7) are directed against flavoenzymes with covalently bound FAD. Clin Exp Immunol 111: 541-547, 1998.
77. Stahle I, Brizzio C, Barile M, Brandsch R. Anti-mitochondrial flavoprotein autoantibodies of patients with myocarditis and dilated cardiomyopathy (anti-M7): interaction with flavin-carrying proteins, effect of vitamin B2 and epitope mapping. Clin Exp Immunol 115: 404-408, 1999.
78. Chen H, Detmer S A, Ewald A J, Griffin E E, Fraser S E, Chan D C. Mitofusins Mfn1 and Mfn2 coordinately regulate mitochondrial fusion and are essential for embryonic development. J Cell Biol 160: 189-200, 2003.
79. Ghebrehiwet B, Ji Y, Valentino A, et al. Soluble gC1qR is an autocrine signal that induces B1R expression on endothelial cells. J Immunol 192: 377-384, 2014.
80. Monlun M, Hyernard C, Blanco P, Lartigue L, Faustin B. Mitochondria as molecular platforms integrating multiple innate immune signalings. J Mol Biol 429: 1-13, 2017.
81. Ellinor I, Tara K, Berhane G. Activation-dependent surface expression of gC1qR/p33 on human blood platelets. Thromb Haemost 89: 331-339, 2003.
82. Xu H, Frojmovic M M, Wong T, Rauch J. p32, a platelet autoantigen recognized by an SLE-derived autoantibody that inhibits platelet aggregation. J Autoimmun 8: 97-119, 1995.
83. Xu H, Frojmovic M M, Wong T, Rauch J. Inhibition of platelet aggregation by an SLE-derived human hybridoma autoantibody against an activation-dependent antigen. Thromb Haemost 74: 1152-1162, 1995.
84. Cozzani E, Drosera M, Gasparini G, Parodi A. Serology of lupus erythematosus: Correlation between immunopathological features and clinical aspects. Autoimmune Dis 2014: 321359 2014.
85. Ortega-Hernandez O D, Shoenfeld Y. Mixed connective tissue disease: An overview of clinical manifestations, diagnosis and treatment. Best Pract Res Clin Rheumatol 26: 61-72, 2012.
86. Alves M R, Isenberg D A. “Mixed connective tissue disease”: a condition in search of an identity. Clin Exp Med 20: 159-166, 2020.
87. Mahler M, Fritzler M J, Satoh M. Autoantibodies to the mitochondrial RNA processing (MRP) complex also known as Th/To autoantigen. Autoimmun Rev 14: 254-257, 2015.
88. Sibani S, LaBaer J. Immunoprofiling using NAPPA protein microarrays. Methods Mol Biol 723: 149-161, 2011.
89. Rekvig, O. P. The dsDNA, Anti-dsDNA Antibody, and Lupus Nephritis: What We Agree on, What Must Be Done, and What the Best Strategy Forward Could Be. Front Immunol 10, 1104, doi:10.3389/fimmu.2019.01104 (2019).

1. A method for identifying a subject having systemic lupus erythematosus (SLE) by detecting whether mitochondrial autoantibodies specific to one or more mitochondrial autoantigenic polypeptide are present; wherein said method comprises:
a. contacting a biological sample from a subject with one or more mitochondrial autoantigenic polypeptide to form a complex between each respective autoantibody and mitochondrial autoantigenic polypeptide;
b. detecting each complex between each respective autoantibody and mitochondrial autoantigenic polypeptide;
c. optionally, measuring the levels of autoantibody specific to one or more mitochondrial autoantigenic polypeptide; and
d. identifying the subject having SLE when one or more autoantibody specific to the one or more mitochondrial autoantigenic polypeptide relative to a control are present;
wherein said one or more mitochondrial autoantigenic polypeptide is or comprises mitofusin-1 (Mfn-1) or C1qBP.

2. A method for the treatment of systemic lupus erythematosus (SLE) in an SLE subject in need thereof, wherein said method comprises:
a. identifying an SLE subject by detecting whether mitochondrial autoantibodies specific to one or more mitochondrial autoantigenic polypeptide are present by a method which comprises:
i. contacting a biological sample from a human subject suspected of suffering from an SLE with one or more mitochondrial autoantigenic polypeptide to form a complex between each respective autoantibody and mitochondrial autoantigenic polypeptide;
ii. detecting each complex between each respective autoantibody and mitochondrial autoantigenic polypeptide;
iii. optionally, measuring the levels of autoantibody specific to one or more mitochondrial autoantigenic polypeptide; and
iv. identifying the SLE subject when one or more autoantibody specific to the one or more mitochondrial autoantigenic polypeptide relative to a control are present; and
b. when the SLE subject is identified, treating SLE subject with anti-SLE therapy;
wherein said one or more mitochondrial autoantigenic polypeptide is or comprises mitofusin-1 (Mfn-1) or C1qBP.

3. A method for diagnosing or determining a progression of systemic lupus erythematosus (SLE) in a subject, said method comprising:
a. providing a biological sample from the subject;
b. detecting one or more autoantibody levels specific to one or more mitochondrial autoantigenic polypeptide; and
c. diagnosing the subject as having SLE or determining the progression of SLE by observing significantly increased autoantibody levels of specific to the one or more mitochondrial autoantigenic polypeptide as compared to a subject not having SLE;
wherein said one or more mitochondrial autoantigenic polypeptide is or comprises mitofusin-1 (Mfn-1) or C1qBP.

4. The method of any one of claims 1 to 3, wherein the systemic lupus erythematosus (SLE) is accompanied by a secondary syndrome or disorder.

5. The method of claim 4, wherein the secondary syndrome or disorder is antiphospholipid syndrome.

6. The method of any one of claims 1 to 5, wherein the detecting of one or more autoantibody/autoantibodies is determined using an immunoassay.

7. The method of any one of claims 1 to 6, wherein the subject is a human.

8. The method of any one of claims 1 to 7, wherein the sample is blood, serum, or plasma.

9. The method of any one of claims 1 to 8, wherein the method further comprises a step of detecting or identifying the levels of one or more autoantibodies selected from the group consisting of anti-nuclear antibodies, anti-double stranded DNA antibodies, lupus anticoagulant antibodies, antiphospholipid antibodies, anticardiolipin antibodies, anti-β2 Glycoprotein I antibodies, anti-ribonucleoprotein antibodies, anti-small nuclear ribonucleoprotein antibodies, anti-Smith (Sm) anti-Ro (SS/A) antibodies, anti-La (SS/B) antibodies, anti-mitochondrial DNA, anti-whole mitochondria, anti-mitochondrial RNA antibodies, and any combination thereof.

10. The method of any one of claims 1 to 9, wherein the method further comprises a step of determining whether one or more criteria selected from the group consisting of American College of Rheumatology (ACR) classification criteria, Systemic Lupus International Collaborating Clinics (SLICC) classification criteria, Systemic Lupus Erythematosus Disease Activity Index (SLEDAI), lupus severity index (LSI), Systemic Lupus International Collaborating Clinics/American College of Rheumatology (SLICC/ACR) Damage Index (SDI), European League Against Rheumatism (EULAR) classification criteria, and criteria selected from FIGS. 10 and/or 11 are present.

11. A method of detecting one or more autoantibodies specific to one or more mitochondrial autoantigenic polypeptide in a biological sample from a human subject suspected of having systemic lupus erythematosus (SLE); said method comprising:
a. contacting the biological sample from the human subject with one or more mitochondrial autoantigenic polypeptide to form a complex between each respective autoantibody and mitochondrial autoantigenic polypeptide;
b. detecting each complex between each respective autoantibody and mitochondrial autoantigenic polypeptide; and
c. optionally, measuring the levels of autoantibody specific to one or more mitochondrial autoantigenic polypeptide;
wherein said one or more mitochondrial autoantigenic polypeptide is or comprises mitofusin-1 (Mfn-1) or C1qBP.

12. The method of claim 11, wherein one or more autoantibodies as defined in claim 14 are present in the human subject suspected of having SLE.

13. The method of claim 11 or 12, wherein one or more criteria as defined in claim 15 are present in the human subject suspected of having SLE.

14. The method of any one of claims 11 to 13, wherein two or more mitochondrial autoantigenic polypeptide are detected.

15. The method of any one of claims 11 to 14, further comprising one or more features as defined in any one of claims 4 to 10.

16. A kit for use in diagnosing or determining the progression of SLE in a subject, said kit comprising one or more reagents for detecting autoantibodies specific to one or more mitochondrial autoantigenic polypeptides in a biological sample from the subject, wherein said one or more mitochondrial autoantigenic polypeptide is or comprises mitofusin-1 (Mfn-1) or C1qBP.

17. The kit of claim 16, wherein the systemic lupus erythematosus (SLE) is accompanied by a secondary syndrome or disorder.

18. The kit of claim 17, wherein the secondary syndrome or disorder is antiphospholipid syndrome.

19. The kit of any one of claims 16 to 18, wherein the detecting of autoantibody levels is determined using an immunoassay.

20. The kit of any one of claims 16 to 19, wherein the subject is a human.

21. The kit of any one of claims 16 to 20, wherein the sample is blood, serum, or plasma.

22. The kit for use of any one of claims 16 to 21, further comprising reagents for detecting one or more autoantibodies known to be associated with the diagnosis or progression of said SLE.

23. The kit for use of any one of claims 16 to 22, wherein the kit is used in combination with the detection of one or more autoantibodies known to be associated with the identification, diagnosis or progression of said SLE.

24. The kit of claim 16 to 23, wherein the one or more autoantibodies known to be associated with the diagnosis or progression of said SLE is/are selected from the group consisting of anti-nuclear antibodies, anti-double stranded DNA antibodies, lupus anticoagulant antibodies, antiphospholipid antibodies, anticardiolipin antibodies, anti-β2 Glycoprotein I antibodies, anti-ribonucleoprotein antibodies, anti-small nuclear ribonucleoprotein antibodies, anti-Ro (SS/A) antibodies, anti-La (SS/B) antibodies, anti-mitochondrial DNA, anti-whole mitochondria, anti-mitochondrial RNA antibodies, and any combination thereof.

25. The kit for use of any one of claims 16 to 24, wherein the kit is used in combination with one or more criteria selected from one or more known diagnostic or classification measures of SLE.

26. The kit of claim 25, wherein the one or more known diagnostic or classification measures is/are selected from the group consisting of American College of Rheumatology (ACR) classification criteria, Systemic Lupus International Collaborating Clinics (SLICC) classification criteria, Systemic Lupus Erythematosus Disease Activity Index (SLEDAI), lupus severity index (LSI), Systemic Lupus International Collaborating Clinics/American College of Rheumatology (SLICC/ACR) Damage Index (SDI), European League Against Rheumatism (EULAR) classification criteria, and criteria selected from FIGS. 10 and/or 11.

27. A method of producing a complex comprising one or more mitochondrial autoantigenic polypeptides and one or more autoantibodies specific to the respective one or more mitochondrial autoantigenic polypeptides; wherein said method comprises:
a. contacting a biological sample from a subject comprising one or more autoantibodies, with one or more mitochondrial autoantigenic polypeptides, wherein the subject has or is suspected to have systemic lupus erythematosus (SLE); and
b. detecting each complex between each respective autoantibody and mitochondrial autoantigenic polypeptide;
wherein said one or more mitochondrial autoantigenic polypeptide is or comprises mitofusin-1 (Mfn-1) or C1qBP.

28. The method of claim 27, further comprising one or more features as defined in any one of claims 4 to 10.

29. A complex for use in the identification, diagnosis, or determination of progression of systemic lupus erythematosus (SLE) in a subject, wherein the complex comprises or is formed by one or more mitochondrial autoantigenic polypeptides and one or more autoantibodies specific to the respective one or more mitochondrial autoantigenic polypeptides, wherein said one or more mitochondrial autoantigenic polypeptide is or comprises mitofusin-1 (Mfn-1) or C1qBP.

30. A complex for use as a research tool in the detection of one or more autoantibodies specific to the one or more mitochondrial autoantigenic polypeptides in a biological sample, wherein the complex comprises one or more mitochondrial autoantigenic polypeptides and one or more autoantibodies specific to the respective one or more mitochondrial autoantigenic polypeptides, wherein said one or more mitochondrial autoantigenic polypeptide is or comprises mitofusin-1 (Mfn-1) or C1qBP.

31. A kit for use in the detection of autoantibodies in a biological sample, wherein the kit comprises autoantigens specific to the autoantibodies and reagents for the detection of the autoantibodies, wherein at least one of the autoantibodies are one or more autoantibodies specific to one or more mitochondrial autoantigenic polypeptides, and wherein at least one of the autoantigens are one or more mitochondrial autoantigenic polypeptides, wherein said one or more mitochondrial autoantigenic polypeptide is or comprises mitofusin-1 (Mfn-1) or C1qBP.

32. A kit for use in the detection of one or more autoantibodies specific to a respective one or more mitochondrial autoantigenic polypeptides in a biological sample, wherein the kit comprises one or more mitochondrial autoantigenic polypeptides specific to the respective one or more autoantibodies and reagents for the detection of the one or more autoantibodies, wherein said one or more mitochondrial autoantigenic polypeptide is or comprises mitofusin-1 (Mfn-1) or C1qBP.

33. A method of manufacturing the kit as defined in any one of claims 16 to 26, or 32, said method comprising:
a. immobilizing the one or more mitochondrial autoantigenic polypeptides; and
b. providing reagents for the detection of autoantibodies specific to the respective one or more mitochondrial autoantigenic polypeptides.

34. A method of manufacturing the kit as defined in claim 31, said method comprising:
a. immobilizing the autoantigens; and
b. providing reagents for the detection of autoantibodies specific to the respective autoantigens.

35. A method for producing or modifying a test for detecting systemic lupus erythematosus, the method comprising adding or integrating into said test quantifying a panel of mitochondrial autoantibodies, the panel comprising mitochondrial autoantibodies specific to one or more mitochondrial autoantigenic polypeptide, wherein said one or more mitochondrial autoantigenic polypeptide is or comprises mitofusin-1 (Mfn-1) or C1qBP.

36. The method of claim 35, further comprising one or more features as defined in any one of claims 4 to 10.
